(19) United States
(12) Patent Application Publication
(10) Pub. No.: US 2026/0334765 A1
(43) Pub. Date: Oct. 29, 2026

(54) ENDOSCOPIC OBJECTIVE LENS AND ENDOSCOPE

(71) Applicant: FUJIFILM Corporation, Tokyo (JP)

(72) Inventor: Tomoyuki BABA, Saitama (JP)

(73) Assignee: FUJIFILM Corporation, Tokyo (JP)

(21) Appl. No.: 19/467,752

(22) Filed: Feb. 2, 2026

(30) Foreign Application Priority Data
Feb. 17, 2025 (JP) 2025-023640

(51) Int. Cl. A61B 1/00; G02B 15/14; G02B 15/177

(52) U.S. Cl. CPC A61B 1/00188; G02B 15/144513; G02B 15/177

(57) ABSTRACT

An endoscopic objective lens consists of, in order from a position closest to an object side to an image side, four or fewer lens groups including a first stationary lens group that does not move during changing magnification and during focusing, a first moving lens group, and a second moving lens group. During changing the magnification, the first moving lens group and the second moving lens group move along different trajectories from each other. For a lens group that moves during the focusing, a moving amount during changing the magnification from a wide angle end state in farthest point observation to a telephoto end state in the farthest point observation is different from a moving amount during the focusing from the wide angle end state in the farthest point observation to a wide angle end state in nearest point observation.

### CROSS-REFERENCE TO RELATED APPLICATIONS

This application claims priority from Japanese Patent Application No. 2025-023640, filed on Feb. 17, 2025, the entire disclosure of which is incorporated herein by reference.

### BACKGROUND

### Technical Field

The disclosed technology relates to an endoscopic objective lens and an endoscope.

### Related Art

In the related art, a lens system according to JP2021-189444A is known as an objective lens that can be used in an endoscope.

### SUMMARY

In order not to miss a lesion portion, an endoscope is required to provide not only wide-range observation but also magnified observation of a lesion portion. In the related art, an endoscopic objective lens that enables magnified observation of a lesion portion by changing a focal length during focusing has been proposed. However, in such an endoscopic objective lens, magnification changing and focusing are performed at the same time. Thus, a range of a visual field changes during the focusing, or a focus position changes during changing the magnification, and this poses an issue of missing a lesion portion. This issue is preferably eliminated by independently performing magnification changing and focusing. However, doing so increases the number of lens groups to be moved. Thus, complexity of a moving mechanism for lenses may be increased, and a diameter of the endoscope may be increased.

The present disclosure provides an endoscopic objective lens capable of independently performing magnification changing and focusing, and reducing an increase in a diameter of an endoscope, and an endoscope using the endoscopic objective lens.

An endoscopic objective lens according to an aspect of the present disclosure consists of, in order from a position closest to an object side to an image side, four or fewer lens groups including a first stationary lens group having optical power, a first moving lens group having optical power, and a second moving lens group having optical power, in which during changing magnification from a wide angle end state in farthest point observation to a telephoto end state in the farthest point observation, the first moving lens group and the second moving lens group move along different trajectories from each other, during focusing from the wide angle end state in the farthest point observation to a wide angle end state in nearest point observation, at least one of the first moving lens group or the second moving lens group moves, for a lens group that moves during the focusing, a moving amount during changing the magnification from the wide angle end state in the farthest point observation to the telephoto end state in the farthest point observation is different from a moving amount during the focusing from the wide angle end state in the farthest point observation to the wide angle end state in the nearest point observation, the first stationary lens group remains stationary with respect to an image plane during changing the magnification and during the focusing, and Conditional Expression (1) is satisfied, which is represented by

0<YFw/(fFw×tan ωFw)<0.5 (1).

A maximum image height in the wide angle end state in the farthest point observation is denoted by YFw. A maximum half angle of view in the wide angle end state in the farthest point observation is denoted by ωFw. A focal length of a whole system in the wide angle end state in the farthest point observation is denoted by fFw.

The endoscopic objective lens of the aspect preferably satisfies Conditional Expression (1-1) represented by

0.02<YFw/(fFw×tan ωFw)<0.3 (1-1), and

more preferably satisfies Conditional Expression (1-2) represented by

0.04<YFw/(fFw×tan ωFw)<0.2 (1-2).

During the focusing from the wide angle end state in the farthest point observation to the wide angle end state in the nearest point observation, the first moving lens group and the second moving lens group may be configured to move along different trajectories from each other.

During the focusing from the wide angle end state in the farthest point observation to the wide angle end state in the nearest point observation, the first moving lens group and the second moving lens group may be configured to move in an integrated manner.

The endoscopic objective lens of the aspect may be configured to further comprise a second stationary lens group that remains stationary with respect to the image plane during changing the magnification and during the focusing and that has optical power, between the first moving lens group and the second moving lens group.

The endoscopic objective lens of the aspect may be configured to further comprise a second stationary lens group that remains stationary with respect to the image plane during changing the magnification and during the focusing and that has optical power, on an image side of the second moving lens group.

In a case where a maximum half angle of view in the telephoto end state in the farthest point observation is denoted by ωFt, it is preferable that the endoscopic objective lens of the aspect satisfies Conditional Expression (2) represented by

1.2<ωFw/ωFt<3 (2).

In a case where a maximum half angle of view in the wide angle end state in the nearest point observation is denoted by ωCw, the endoscopic objective lens of the aspect preferably satisfies Conditional Expression (3) represented by

0.5<ωFw/ωCw<2 (3), and

more preferably satisfies Conditional Expression (3-1) represented by

0.9<ωFw/ωCw<1.2 (3-1).

In a case where a distance on an optical axis from a farthest point in the wide angle end state in the farthest point observation to a surface closest to the object side of the first stationary lens group is denoted by D0Fw, a focal length of the whole system in the wide angle end state in the nearest point observation is denoted by fCw, and a distance on the optical axis from a nearest point in the wide angle end state in the nearest point observation to the surface closest to the object side of the first stationary lens group is denoted by D0Cw, the endoscopic objective lens of the aspect preferably satisfies Conditional Expression (4) represented by

−0.1<(fCw−fFw)/(D0Fw−D0Cw)<0.1 (4), and
more preferably satisfies Conditional Expression (4-1) represented by

−0.01<(fCw−fFw)/(D0Fw−D0Cw)<0.01 (4-1).

It is preferable that the first stationary lens group includes, in order from the object side to the image side, a first negative lens, a second negative lens, and a first positive lens, and the second negative lens and the first positive lens are cemented to each other.

It is preferable that a lens surface of the first negative lens on the object side is a plane.

In a case where a focal length of the first negative lens is denoted by fL1, the endoscopic objective lens of the aspect preferably satisfies Conditional Expression (5) represented by

0.5<|fL1/fFw|<5 (5),

more preferably satisfies Conditional Expression (5-1) represented by

0.7<|fL1/fFw|<3 (5-1), and

further preferably satisfies Conditional Expression (5-2) represented by

0.8<|fL1/fFw|<2 (5-2).

In a case where a focal length of the first stationary lens group is denoted by f1, and a focal length of a lens group closest to the image side in the endoscopic objective lens is denoted by fE, the endoscopic objective lens of the aspect preferably satisfies Conditional Expression (6) represented by

0.05<|f1|/fE<2.3 (6), and

more preferably satisfies Conditional Expression (6-1) represented by

0.15<|f1|/fE<1.2 (6-1).

Another aspect of the present disclosure is an endoscope comprising the endoscopic objective lens of the aspect.

In the present specification, “consisting of” or “consists of” means that a lens substantially not having optical power, an optical element, other than a lens, such as a stop, a filter, and a cover glass, a lens flange, a lens barrel, an imaging element, and the like may be included in addition to the illustrated constituents.

In the present specification, a “group having positive optical power” means that the whole group has positive optical power. Similarly, in the present specification, a “group having negative optical power” means that the whole group has negative optical power. A “lens having positive optical power” and a “positive lens” are synonymous with each other. A “lens having negative optical power” and a “negative lens” are synonymous with each other. A “lens group” is not limited to a configuration consisting of a plurality of lenses and may be configured to consist of only one lens.

In the present specification, the number of lenses is the number of lenses as constituents. For example, the number of lenses in a cemented lens in which a plurality of single lenses formed of different materials are cemented is represented by the number of single lenses constituting the cemented lens. A compound aspherical lens (a lens in which a lens (for example, a spherical lens) and an aspherical surface-shaped film formed on the lens are configured to be integrated with each other and that functions as one aspherical lens as a whole) is not regarded as a cemented lens and is treated as one lens. Unless otherwise specified, a sign of optical power and a surface shape in a paraxial region are used with respect to a lens including an aspherical surface.

In the present specification, a phrase “including, in order from the object side to the image side” includes both a case of consecutively including constituents in order and a case of non-consecutively including constituents in order. For example, in the present specification, “including, in order from the object side to the image side, A and B” means that A and B may be consecutively disposed, or other objects may be disposed between A and B as long as B is disposed on the image side with respect to A.

In the present specification, a “whole system” refers to the endoscopic objective lens. A “focal length” used in the conditional expressions is a paraxial focal length. Unless otherwise specified, a “distance on the optical axis” used in the conditional expressions is a geometrical distance. Values used in the conditional expressions are values based on a d line. A “lens group” may include, for example, optical elements other than a lens, such as a parallel plate and/or a stop. A phrase “moves on different trajectories from each other” related to movement of a lens group is synonymous with “moves by changing a mutual spacing”.

A “d line”, a “C line”, an “F line”, and an “h line” described in the present specification are bright lines. A wavelength of the d line is 587.56 nanometers (nm). A wavelength of the C line is 656.27 nanometers (nm). A wavelength of the F line is 486.13 nanometers (nm). A wavelength of the h line is 404.66 nanometers (nm).

According to the present disclosure, an endoscopic objective lens capable of independently performing magnification changing and focusing, and reducing an increase in a diameter of an endoscope, and an endoscope using the endoscopic objective lens can be provided.

### BRIEF DESCRIPTION OF THE DRAWINGS

FIG. 1 is a cross-sectional view corresponding to an endoscopic objective lens of Example 1, showing a configuration of an endoscopic objective lens according to an embodiment.

FIG. 2 is a cross-sectional view showing a configuration and luminous fluxes in each state of the endoscopic objective lens of Example 1.

FIG. 3 is each aberration diagram of the endoscopic objective lens of Example 1.

FIG. 4 is a cross-sectional view showing a configuration of an endoscopic objective lens of Example 2.

FIG. 5 is each aberration diagram of the endoscopic objective lens of Example 2.

FIG. 6 is a cross-sectional view showing a configuration of an endoscopic objective lens of Example 3.

FIG. 7 is each aberration diagram of the endoscopic objective lens of Example 3.

FIG. 8 is a cross-sectional view showing a configuration of an endoscopic objective lens of Example 4.

FIG. 9 is each aberration diagram of the endoscopic objective lens of Example 4.

FIG. 10 is a cross-sectional view showing a configuration of an endoscopic objective lens of Example 5.

FIG. 11 is each aberration diagram of the endoscopic objective lens of Example 5.

FIG. 12 is a cross-sectional view showing a configuration of an endoscopic objective lens of Example 6.

FIG. 13 is each aberration diagram of the endoscopic objective lens of Example 6.

FIG. 14 is a cross-sectional view showing a configuration of an endoscopic objective lens of Example 7.

FIG. 15 is each aberration diagram of the endoscopic objective lens of Example 7.

FIG. 16 is a cross-sectional view showing a configuration of an endoscopic objective lens of Example 8.

FIG. 17 is each aberration diagram of the endoscopic objective lens of Example 8.

FIG. 18 is a cross-sectional view showing a configuration of an endoscopic objective lens of Example 9.

FIG. 19 is each aberration diagram of the endoscopic objective lens of Example 9.

FIG. 20 is a cross-sectional view showing a configuration of an endoscopic objective lens of Example 10.

FIG. 21 is each aberration diagram of the endoscopic objective lens of Example 10.

FIG. 22 is a cross-sectional view showing a configuration of an endoscopic objective lens of Example 11.

FIG. 23 is each aberration diagram of the endoscopic objective lens of Example 11.

FIG. 24 is a cross-sectional view showing a configuration of an endoscopic objective lens of Example 12.

FIG. 25 is each aberration diagram of the endoscopic objective lens of Example 12.

FIG. 26 is a cross-sectional view showing a configuration of an endoscopic objective lens of Example 13.

FIG. 27 is each aberration diagram of the endoscopic objective lens of Example 13.

FIG. 28 is a schematic configuration diagram of an endoscope according to an embodiment.

### DETAILED DESCRIPTION

Hereinafter, embodiments of the present disclosure will be described with reference to the drawings.

In the following description, “farthest point observation” refers to a state where an endoscopic objective lens focuses on a farthest point, and “nearest point observation” refers to a state where the endoscopic objective lens focuses on a nearest point. The “farthest point” refers to a point farthest from a surface closest to an object side of the endoscopic objective lens among points on which the endoscopic objective lens can focus. The “nearest point” refers to a point nearest to the surface closest to the object side of the endoscopic objective lens among the points on which the endoscopic objective lens can focus.

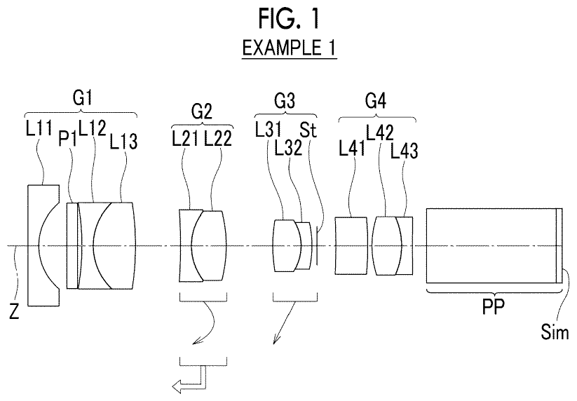

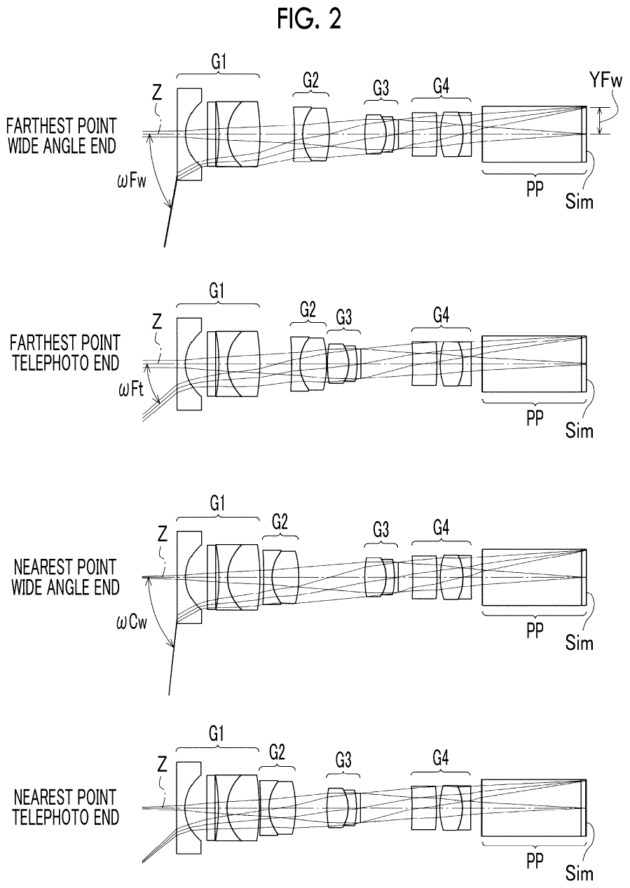

FIG. 1 shows a configuration of the endoscopic objective lens according to an embodiment of the present disclosure in a cross section including an optical axis Z. FIG. 2 shows a cross-sectional view of a configuration and luminous fluxes in each state of the endoscopic objective lens in FIG. 1. In FIG. 2, an uppermost part labeled “FARTHEST POINT WIDE ANGLE END” shows a wide angle end state in the farthest point observation. A second part from the top labeled “FARTHEST POINT TELEPHOTO END” shows a telephoto end state in the farthest point observation. A third part from the top labeled “NEAREST POINT WIDE ANGLE END” shows a wide angle end state in the nearest point observation. A lowermost part labeled “NEAREST POINT TELEPHOTO END” shows a telephoto end state in the nearest point observation. FIG. 2 shows an on-axis luminous flux and a luminous flux at a maximum half angle of view in each state as luminous fluxes. In FIGS. 1 and 2, a left side is the object side, and a right side is an image side. The example shown in FIGS. 1 and 2 corresponds to Example 1 described later.

For example, the endoscopic objective lens in FIG. 1 consists of, in order from the object side to the image side, four lens groups including a first lens group G1, a second lens group G2, a third lens group G3, and a fourth lens group G4. For example, each group in the example in FIG. 1 is configured as follows. The first lens group G1 consists of, in order from the object side to the image side, a lens L11, an optical member P1, a lens L12, and a lens L13. The second lens group G2 consists of, in order from the object side to the image side, a lens L21 and a lens L22. The third lens group G3 consists of, in order from the object side to the image side, a lens L31, a lens L32, and an aperture stop St. The fourth lens group G4 consists of, in order from the object side to the image side, a lens L41, a lens L42, and a lens L43. The optical member P1 is assumed to be a filter and the like and is a member that has an incident surface and an emission surface parallel to each other, and does not have optical power. The aperture stop St in FIG. 1 does not show a size or a shape and shows a position on the optical axis.

In the example in FIG. 1, an optical member PP having an incident surface and an emission surface parallel to each other is disposed between the endoscopic objective lens and an image plane Sim. The optical member PP is a member that is assumed to be a prism, a filter, a cover glass, and the like. The optical member PP is a member not having optical power. A configuration without the optical member PP can also be adopted.

In the example in FIG. 1, the first lens group G1 corresponds to a first stationary lens group of the present disclosure. The second lens group G2 corresponds to a first moving lens group of the present disclosure. The third lens group G3 corresponds to a second moving lens group of the present disclosure. The fourth lens group G4 corresponds to a second stationary lens group of the present disclosure.

The endoscopic objective lens of the present disclosure consists of four or fewer lens groups that are disposed in order from a position closest to the object side to the image side along the optical axis Z and include the first stationary lens group having optical power, the first moving lens group having optical power, and the second moving lens group having optical power. During changing the magnification from the wide angle end state in the farthest point observation to the telephoto end state in the farthest point observation, the first moving lens group and the second moving lens group move along different trajectories from each other. During focusing from the wide angle end state in the farthest point observation to the wide angle end state in the nearest point observation, at least one of the first moving lens group or the second moving lens group moves. For a lens group that moves during the focusing, a moving amount of the lens group during changing the magnification from the wide angle end state in the farthest point observation to the telephoto end state in the farthest point observation is different from a moving amount of the lens group during the focusing from the wide angle end state in the farthest point observation to the wide angle end state in the nearest point observation. Here, the “moving amount” also reflects signs such that a moving amount to the object side is a negative value, and a moving amount to the image side is a positive value.

According to the disclosed technology as described above, the lens group that moves during the focusing is also moved and used during changing the magnification. Thus, the number of moving lens groups can be minimized. Accordingly, an increase in complexity of a moving mechanism can be prevented, and an increase in a diameter of an endoscope can be reduced. In addition, in the disclosed technology, the lens group that moves during changing the magnification and focusing is configured to move along different trajectories between changing the magnification and focusing. That is, the endoscopic objective lens of the present disclosure can independently perform magnification changing and focusing. Accordingly, since a change in a range of a visual field during the focusing and a change in a focus position during changing the magnification can be reduced, an issue of missing a lesion portion during the focusing and/or during changing the magnification can be reduced.

Meanwhile, in a case where magnification changing and focusing are independently performed in the endoscopic objective lens of the related art, at least two lens groups move during changing the magnification, and at least one lens group moves during the focusing. Thus, a total of three or more moving lens groups are required. In a case where the number of moving lens groups is large, the complexity of the moving mechanism for lenses may be increased, and the diameter of the endoscope may be increased. In a case where the diameter of the endoscope is increased, a physical burden on a patient during examination is increased, which is not preferable.

In the endoscopic objective lens of the present disclosure, magnification changing and focusing can be independently performed with only two moving lens groups. Thus, the moving mechanism can be simplified compared to that of the endoscopic objective lens of the related art, and reduction in a size and reduction in a diameter of an optical system can be implemented. That is, according to the endoscopic objective lens of the present disclosure, the issue of missing a lesion portion during the focusing and during changing the magnification can be reduced while establishing compatibility between wide-range observation and magnified observation. Furthermore, an increase in the diameter of the endoscope caused by an increase in the complexity of the moving mechanism can be prevented.

Specifically, for example, in the example in FIG. 1, during changing the magnification, the second lens group G2 and the third lens group G3 move along different trajectories from each other, and other lens groups remain stationary with respect to the image plane Sim. In addition, in the example in FIG. 1, during the focusing, only the second lens group G2 moves, and other lens groups remain stationary with respect to the image plane Sim.

In FIG. 1, brackets and solid arrows are shown below the lens group that moves during changing the magnification. The solid arrow indicates a schematic moving direction of each lens group that moves during changing the magnification from the wide angle end state in the farthest point observation to the telephoto end state in the farthest point observation. In addition, in FIG. 1, a bracket and an outlined arrow are shown below the lens group that moves during the focusing. The outlined arrow indicates a moving direction of each lens group that moves during the focusing from the wide angle end state in the farthest point observation to the wide angle end state in the nearest point observation.

The same illustration method of the moving lens group described above applies to the drawings of other examples described later. The drawings of the present application show that a plurality of constituents illustrated with one bracket connected to an arrow indicating movement move in an integrated manner. Here, “moving in an integrated manner” means moving at the same time in the same direction by the same amount.

In the example in FIG. 1, the first lens group G1 and the fourth lens group G4 remain stationary with respect to the image plane Sim during changing the magnification and during the focusing. In the disclosed technology, the first stationary lens group disposed closest to the object side remains stationary with respect to the image plane Sim during changing the magnification and during the focusing. Causing the lens group closest to the object side to remain stationary provides an advantage in securing airtightness. In the endoscope, the endoscopic objective lens is generally mounted on the endoscope without a protective member so that a lens closest to the object side in the endoscopic objective lens also functions as an optical window. In this case, securing airtightness is required. Thus, a configuration in which the first stationary lens group does not move is advantageous.

The example shown in FIG. 1 is merely an example, and various modifications can be made to the endoscopic objective lens of the present disclosure without departing from the gist of the disclosed technology. For example, the number of lenses included in each lens group may be different from the number in the example in FIG. 1. In addition, configurations of lenses included in each lens group can be different from the configurations in the example in FIG. 1. Furthermore, the lens group that moves during changing the magnification, and the lens group that moves during the focusing can have different configurations from the example in FIG. 1.

For example, in the example in FIG. 1, only one lens group moves during the focusing. However, in the disclosed technology, during the focusing from the wide angle end state in the farthest point observation to the wide angle end state in the nearest point observation, the first moving lens group and the second moving lens group may be configured to move along different trajectories. Doing so provides an advantage in securing more favorable optical performance in the whole range of magnification changing and focusing.

Alternatively, during the focusing from the wide angle end state in the farthest point observation to the wide angle end state in the nearest point observation, the first moving lens group and the second moving lens group may be configured to move in an integrated manner. Doing so provides an advantage in simplifying the moving mechanism while securing more favorable optical performance in the whole range of magnification changing and focusing.

The endoscopic objective lens of the present disclosure may be configured to include, between the first moving lens group and the second moving lens group, the second stationary lens group that remains stationary with respect to the image plane Sim during changing the magnification and during the focusing and that has optical power. Doing so provides an advantage in securing more favorable optical performance in the whole range of magnification changing and focusing.

Alternatively, the endoscopic objective lens of the present disclosure may be configured to include, on the image side of the second moving lens group, the second stationary lens group that remains stationary with respect to the image plane Sim during changing the magnification and during the focusing and that has optical power. Doing so provides an advantage in securing more favorable optical performance in the whole range of magnification changing and focusing.

Alternatively, the endoscopic objective lens of the present disclosure may be configured to consist of three lens groups including the first stationary lens group, the first moving lens group, and the second moving lens group. Doing so provides an advantage in reducing a size of a lens system.

The first stationary lens group may be configured to include, in order from the object side to the image side, a first negative lens having negative optical power, a second negative lens having negative optical power, and a first positive lens having positive optical power. The second negative lens and the first positive lens may be configured to be cemented to each other. Doing so provides an advantage in reducing axial chromatic aberration and lateral chromatic aberration from a visible range to a short wavelength range near a wavelength of 400 nanometers (nm) while performing observation with a wide angle of view.

It is preferable that a surface of the first negative lens on the object side is a plane. Doing so provides an advantage in reducing an increase in an outer diameter of the first negative lens and also provides an advantage in improving manufacturability of the first negative lens. The first negative lens may be disposed closest to the object side. In this case, forming the surface of the first negative lens on the object side to be a plane can reduce application of a liquid or the like to the surface of the first negative lens on the object side. Here, the term “plane” includes a range of error generally allowed in the technical field.

The number of lenses included in the first moving lens group may be configured to be two or fewer. Doing so provides an advantage in reducing the size of the lens system and simplifying the moving mechanism. In a case where the number of lenses included in the first moving lens group is two, the two lenses may be configured to be a negative lens and a positive lens cemented to each other. Doing so provides an advantage in reducing a change in chromatic aberration during changing the magnification.

The second moving lens group may be configured to include a cemented lens in which a positive lens and a negative lens are cemented to each other. Doing so provides an advantage in reducing a change in chromatic aberration during changing the magnification.

Next, preferable configurations related to conditional expressions of the endoscopic objective lens of the present disclosure will be described. In the following description related to the conditional expressions, to avoid redundant description, the same symbols will be used for the same definitions to omit duplicate descriptions of symbols. In addition, to avoid redundant description, the “endoscopic objective lens of the present disclosure” will be simply referred to as the “endoscopic objective lens” below.

It is preferable that the endoscopic objective lens satisfies Conditional Expression (1). A maximum image height in the wide angle end state in the farthest point observation is denoted by YFw. A maximum half angle of view in the wide angle end state in the farthest point observation is denoted by ωFw. A focal length of a whole system in the wide angle end state in the farthest point observation is denoted by fFw. Here, tan denotes a tangent. For example, FIG. 2 shows the maximum image height YFw and the maximum half angle of view ωFw. Since YFw>0, fFw>0, and tan ωFw>0 are established, 0<YFw/(fFw×tan ωFw) is established for a lower limit of Conditional Expression (1). Ensuring that a corresponding value of Conditional Expression (1) is not greater than or equal to its upper limit value facilitates increasing a magnification near a center of an image forming region while performing observation with a wide angle of view.

0<YFw/(fFw×tan ωFw)<0.5 (1)

It is more preferable that the endoscopic objective lens satisfies Conditional Expression (1-1). Ensuring that a corresponding value of conditional expression (1-1) is not less than or equal to its lower limit value can reduce an increase in an outer diameter of the lens. To obtain more favorable characteristics, it is preferable that the endoscopic objective lens satisfies Conditional Expression (1-2).

0.02<YFw/(fFw×tan ωFw)<0.3 (1-1)

0.04<YFw/(fFw×tan ωFw)<0.2 (1-2)

In a case where a maximum half angle of view in the telephoto end state in the farthest point observation is denoted by ωFt, it is preferable that the endoscopic objective lens satisfies Conditional Expression (2). For example, FIG. 2 shows the maximum half angle of view ωFt. Ensuring that a corresponding value of Conditional Expression (2) is not less than or equal to its lower limit value facilitates observation of a lesion portion or the like in a more magnified manner during changing the magnification. Ensuring that the corresponding value of Conditional Expression (2) is not greater than or equal to its upper limit value can reduce an increase in the outer diameter and/or a total length of the lens.

1.2<ωFw/ωFt<3 (2)

In a case where a maximum half angle of view in the wide angle end state in the nearest point observation is denoted by ωCw, it is preferable that the endoscopic objective lens satisfies Conditional Expression (3). For example, FIG. 2 shows the maximum half angle of view ωCw. Satisfying Conditional Expression (3) prevents the angle of view from significantly changing during the focusing and thus, facilitates observation of an object to be observed without missing it.

0.5<ωFw/ωCw<2 (3)

To obtain more favorable characteristics, it is preferable that the endoscopic objective lens satisfies Conditional Expression (3-1).

0.9<ωFw/ωCw<1.2 (3-1)

It is preferable that the endoscopic objective lens satisfies Conditional Expression (4). Here, a distance on the optical axis from the farthest point in the wide angle end state in the farthest point observation to a surface closest to the object side of the first stationary lens group is denoted by D0Fw. A focal length of the whole system in the wide angle end state in the nearest point observation is denoted by fCw. A distance on the optical axis from the nearest point in the wide angle end state in the nearest point observation to the surface closest to the object side of the first stationary lens group is denoted by D0Cw. Satisfying Conditional Expression (4) facilitates focusing in a wider range without significantly changing the angle of view.

−0.1<(fCw−fFw)/(D0Fw−D0Cw)<0.1 (4)

To obtain more favorable characteristics, it is preferable that the endoscopic objective lens satisfies Conditional Expression (4-1).

−0.01<(fCw−fFw)/(D0Fw−D0Cw)<0.01 (4-1)

In a configuration in which the first stationary lens group includes, in order from the object side to the image side, the first negative lens, the second negative lens, and the first positive lens, it is preferable that the endoscopic objective lens satisfies Conditional Expression (5). A focal length of the first negative lens is denoted by fL1. Ensuring that a corresponding value of Conditional Expression (5) is not less than or equal to its lower limit value provides an advantage in securing favorable optical performance in the whole range of magnification changing and focusing. Ensuring that the corresponding value of Conditional Expression (5) is not greater than or equal to its upper limit value provides an advantage in reducing an increase in the size of the lens system.

0.5<|fL1/fFw|<5 (5)

To obtain more favorable characteristics, the endoscopic objective lens preferably satisfies Conditional Expression (5-1) and more preferably satisfies Conditional Expression (5-2).

0.7<|fL1/fFw|<3 (5-1)

0.8<|fL1/fFw|<2 (5-2)

It is preferable that the endoscopic objective lens satisfies Conditional Expression (6). A focal length of the first stationary lens group is denoted by f1. A focal length of a lens group closest to the image side in the endoscopic objective lens is denoted by fE. Ensuring that a corresponding value of Conditional Expression (6) is not less than or equal to its lower limit value provides an advantage in reducing an optical total length. Ensuring that the corresponding value of Conditional Expression (6) is not greater than or equal to its upper limit value can reduce overcorrection of spherical aberration.

0.05<|f1|/fE<2.3 (6)

To obtain more favorable characteristics, the endoscopic objective lens preferably satisfies Conditional Expression (6-1) and more preferably satisfies Conditional Expression (6-2).

0.15<|f1|/fE<1.2 (6-1)

0.25<|f1|/fE<0.8 (6-2)

The above preferable configurations and available configurations including the configurations related to the conditional expressions can be adopted in any combination without contradiction and are preferably appropriately selected and adopted in accordance with required specifications.

For example, according to a preferable aspect of the present disclosure, the endoscopic objective lens consists of four or fewer lens groups that are disposed in order from a position closest to the object side to the image side and that include the first stationary lens group having optical power, the first moving lens group having optical power, and the second moving lens group having optical power, in which during changing the magnification from the wide angle end state in the farthest point observation to the telephoto end state in the farthest point observation, the first moving lens group and the second moving lens group move along different trajectories from each other, during the focusing from the wide angle end state in the farthest point observation to the wide angle end state in the nearest point observation, at least one of the first moving lens group or the second moving lens group moves, for a lens group that moves during the focusing, a moving amount during changing the magnification from the wide angle end state in the farthest point observation to the telephoto end state in the farthest point observation is different from a moving amount during the focusing from the wide angle end state in the farthest point observation to the wide angle end state in the nearest point observation, the first stationary lens group remains stationary with respect to the image plane Sim during changing the magnification and during the focusing, and Conditional Expression (1) is satisfied.

Next, examples of the endoscopic objective lens of the present disclosure will be described with reference to the drawings. To avoid an increase in complexity of the description and the drawings caused by an increasing number of digits of reference numerals, reference numerals given to lenses and lens groups in cross-sectional views of each example are independently used for each example. Accordingly, a common reference numeral given in the drawings of different examples does not necessarily indicate a common configuration.

### Example 1

A cross-sectional view showing a configuration of the endoscopic objective lens of Example 1 is shown in FIGS. 1 and 2, and its illustration method is described above. Thus, duplicate descriptions will be omitted in part. The endoscopic objective lens of Example 1 consists of, in order from the object side to the image side, four lens groups including the first lens group G1 having negative optical power, the second lens group G2 having negative optical power, the third lens group G3 having positive optical power, and the fourth lens group G4 having positive optical power.

The first lens group G1 corresponds to the first stationary lens group. The second lens group G2 corresponds to the first moving lens group. The third lens group G3 corresponds to the second moving lens group. The fourth lens group G4 corresponds to the second stationary lens group.

During changing the magnification from the wide angle end to the telephoto end, the second lens group G2 moves to the image side and then moves to the object side, the third lens group G3 moves to the object side, and other lens groups remain stationary with respect to the image plane Sim. During the focusing from the farthest point observation to the nearest point observation, the second lens group G2 moves to the object side, and other lens groups remain stationary with respect to the image plane Sim.

For the endoscopic objective lens of Example 1, Table 1 shows basic lens data, and Table 2 shows specifications and variable surface spacings.

The table of the basic lens data is described as follows. A column of “Sn” shows surface numbers in a case where a surface closest to the object side is a first surface, and the number is increased by one at a time to the image side. A column of “R” shows a curvature radius of each surface. A column of “D” shows a surface spacing on the optical axis between each surface and a surface adjacent thereto on the image side. A column of “Nd” shows a refractive index at a d line for each lens. A column of “vd” shows an Abbe number based on the d line for each lens. A column of “Material” shows a material name of each lens before a period. Here, “OHARA” after a period in the column of “Material” indicates that a manufacturer of the material is OHARA INC.

In the table of the basic lens data, a sign of the curvature radius of a surface having a convex shape facing the object side is positive, and a sign of the curvature radius of a surface having a convex shape facing the image side is negative. A field of the surface number of a surface corresponding to the aperture stop St shows the surface number and a text (St). A value in a lowermost field of the column of D in the table is a spacing between a surface closest to the image side in the table and the image plane Sim. A symbol DD[ ] is used for the variable surface spacings. A surface number on the object side of a spacing is shown in [ ] in the column of the surface spacing.

Table 2 shows an object distance, a zoom ratio, a focal length, an F-number, a maximum full angle of view, a maximum image height, and variable surface spacings based on a d line. In the farthest point observation, the object distance is a distance on the optical axis from the farthest point to a lens surface closest to the object side. In the nearest point observation, the object distance is a distance on the optical axis from the nearest point to the lens surface closest to the object side. The maximum full angle of view is twice the maximum half angle of view. In a field of the maximum full angle of view, [°] indicates a degree unit. In Table 2, a column of “Farthest Point/Wide Angle End” shows each value in the wide angle end state in the farthest point observation. A column of “Farthest Point/Telephoto End” shows each value in the telephoto end state in the farthest point observation. A column of “Nearest Point/Wide Angle End” shows each value in the wide angle end state in the nearest point observation. A column of “Nearest Point/Telephoto End” shows each value in the telephoto end state in the nearest point observation.

In the data of each table, a degree unit is used for angles, and a millimeter (mm) unit is used for lengths. However, since the optical system can also be proportionally enlarged or proportionally reduced and used, other appropriate units can also be used. Each table below shows numerical values rounded to predetermined digits.

TABLE 1
Example 1

| Sn | R | D | Nd | νd | Material |
|---|---|---|---|---|---|
| 1 | ∞ | 0.2500 | 2.00100 | 29.14 | S-LAH99.OHARA |
| 2 | 1.2903 | 0.6498 | | | |
| 3 | ∞ | 0.2500 | 2.00100 | 29.14 | S-LAH99.OHARA |
| 4 | ∞ | 0.1000 | | | |
| 5 | −5.7303 | 0.2600 | 1.71300 | 53.87 | S-LAL8.OHARA |
| 6 | 1.3687 | 0.9773 | 1.84666 | 23.78 | S-TIH53W.OHARA |
| 7 | −6.6664 | DD[7] | | | |
| 8 | −11.7581 | 0.2500 | 1.95375 | 32.32 | S-LAH98.OHARA |
| 9 | 1.2965 | 0.8064 | 1.62004 | 36.26 | S-TIM2.OHARA |
| 10 | −3.2077 | DD[10] | | | |
| 11 | 3.5231 | 0.6444 | 1.59522 | 67.73 | S-FPM2.OHARA |
| 12 | −1.1194 | 0.2500 | 1.62004 | 36.26 | S-TIM2.OHARA |
| 13 | −2.5408 | 0.1173 | | | |
| 14 (St) | ∞ | DD[14] | | | |
| 15 | −12.4672 | 0.7356 | 1.95906 | 17.47 | S-NPH3.OHARA |
| 16 | −8.2170 | 0.1000 | | | |
| 17 | 2.1716 | 0.6854 | 1.48749 | 70.24 | S-FSL5.OHARA |
| 18 | −1.6375 | 0.2500 | 1.88300 | 40.76 | S-LAH58.OHARA |
| 19 | 82.9126 | 0.3272 | | | |
| 20 | ∞ | 2.9961 | 1.74320 | 49.34 | S-LAM60.OHARA |
| 21 | ∞ | 0.1400 | 1.51633 | 64.14 | S-BSL7.OHARA |
| 22 | ∞ | 0.0000 | | | |

TABLE 2
Example 1

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.50 | 0.95 | 1.35 |
| Focal Length | 0.817 | 1.222 | 0.772 | 1.103 |
| F-Number | 4.12 | 5.30 | 4.05 | 5.06 |
| Maximum Full Angle | 159.6 | 81.8 | 167.8 | 82.8 |
| of View[°] | | | | |
| Maximum Image Height | 0.832 | 0.832 | 0.832 | 0.832 |
| DD[7] | 1.0425 | 0.9634 | 0.1195 | 0.0404 |
| DD[10] | 1.0753 | 0.0261 | 1.9983 | 0.9491 |
| DD[14] | 0.4356 | 1.5639 | 0.4356 | 1.5639 |

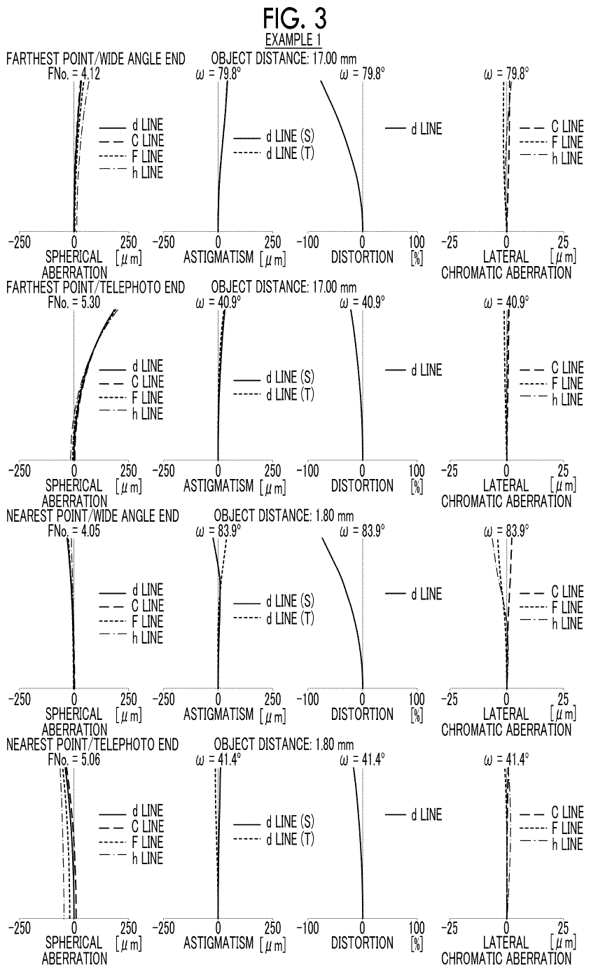

FIG. 3 shows each aberration diagram of the endoscopic objective lens of Example 1. FIG. 3 shows, in order from the left, a spherical aberration diagram, an astigmatism diagram, a distortion diagram, and a lateral chromatic aberration diagram. In FIG. 3, an uppermost part labeled “FARTHEST POINT/WIDE ANGLE END” shows each aberration diagram in the wide angle end state in the farthest point observation. A second part from the top labeled “FARTHEST POINT/TELEPHOTO END” shows each aberration diagram in the telephoto end state in the farthest point observation. A third part from the top labeled “NEAREST POINT/WIDE ANGLE END” shows each aberration diagram in the wide angle end state in the nearest point observation. A lowermost part labeled “NEAREST POINT/TELEPHOTO END” shows each aberration diagram in the telephoto end state in the nearest point observation. In addition, FIG. 3 shows a value of the object distance in millimeter (mm) units on the right of “OBJECT DISTANCE:”. In the spherical aberration diagram, aberration at a d line, a C line, an F line, and an h line is shown by a solid line, a long broken line, a short broken line, and a dot-dashed line, respectively. In the astigmatism diagram, aberration in a sagittal direction at a d line is shown by a solid line, and aberration in a tangential direction at a d line is shown by a short broken line. In the distortion diagram, aberration at a d line is shown by a solid line. In the lateral chromatic aberration diagram, aberration at a C line, an F line, and an h line is shown by a long broken line, a short broken line, and a dot-dashed line, respectively. In the spherical aberration diagram, a value of the F-number in each state is shown after “FNo.=”. In other aberration diagrams, a value of the maximum half angle of view in each state is shown after “ω=”.

The same symbols, meanings, description methods, and illustration methods of each data related to Example 1 apply to the following examples unless otherwise specified. Thus, duplicate descriptions will be omitted below.

### Example 2

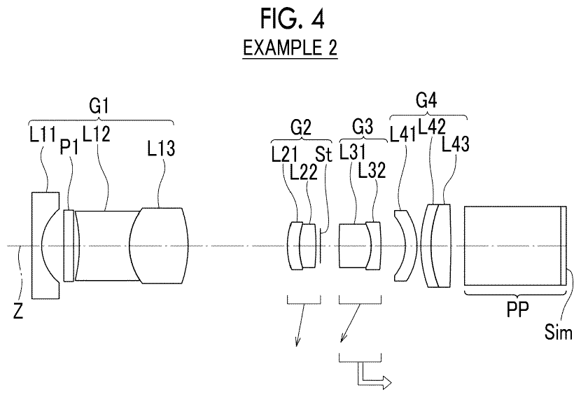

A cross-sectional view of a configuration of an endoscopic objective lens of Example 2 is shown in FIG. 4. The endoscopic objective lens of Example 2 consists of, in order from the object side to the image side, four lens groups including the first lens group G1 having negative optical power, the second lens group G2 having positive optical power, the third lens group G3 having negative optical power, and the fourth lens group G4 having positive optical power.

The first lens group G1 consists of, in order from the object side to the image side, the lens L11, the optical member P1, the lens L12, and the lens L13. The second lens group G2 consists of, in order from the object side to the image side, the lens L21, the lens L22, and the aperture stop St. The third lens group G3 consists of, in order from the object side to the image side, the lens L31 and the lens L32. The fourth lens group G4 consists of, in order from the object side to the image side, the lens L41, the lens L42, and the lens L43. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second moving lens group. The fourth lens group G4 corresponds to the second stationary lens group.

During changing the magnification from the wide angle end to the telephoto end, the second lens group G2 and the third lens group G3 move to the object side along different trajectories from each other, and other lens groups remain stationary with respect to the image plane Sim. During the focusing from the farthest point observation to the nearest point observation, the third lens group G3 moves to the image side, and other lens groups remain stationary with respect to the image plane Sim.

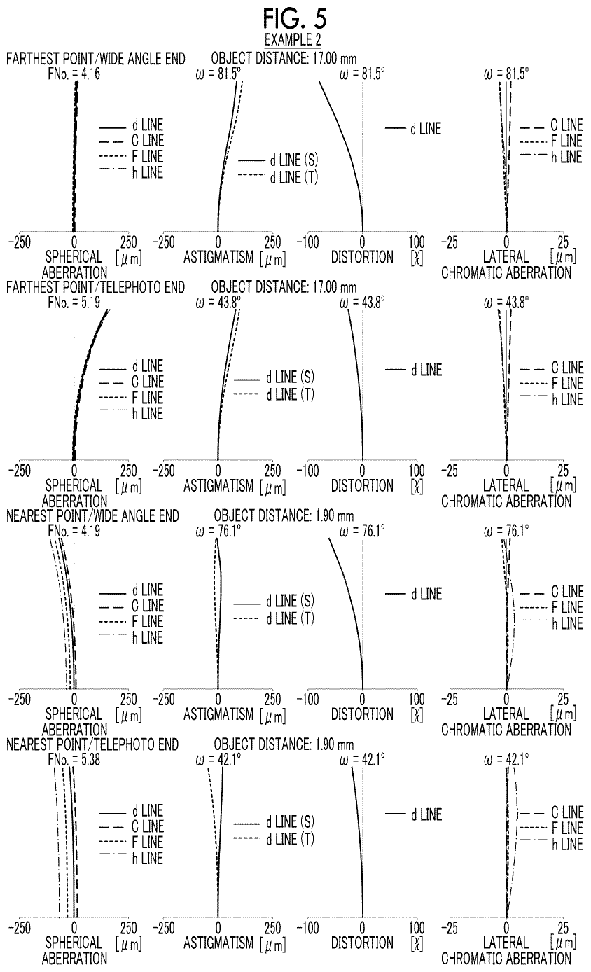

For the endoscopic objective lens of Example 2, Table 3 shows basic lens data, Table 4 shows specifications and variable surface spacings, and FIG. 5 shows each aberration diagram.

TABLE 3
Example 2

| Sn | R | D | Nd | νd | Material |
|---|---|---|---|---|---|
| 1 | ∞ | 0.2500 | 1.88300 | 40.76 | S-LAH58.OHARA |
| 2 | 1.2201 | 0.5701 | | | |
| 3 | ∞ | 0.2500 | 2.00100 | 29.14 | S-LAH99.OHARA |
| 4 | ∞ | 0.1500 | | | |
| 5 | −3.6739 | 1.2872 | 1.89190 | 37.13 | S-LAH92.OHARA |
| 6 | 1.5712 | 1.5100 | 1.72825 | 28.46 | S-TIH10.OHARA |
| 7 | −3.0120 | DD[7] | | | |
| 8 | 1.9438 | 0.2954 | 1.48749 | 70.24 | S-FSL5.OHARA |
| 9 | 1.9309 | 0.4287 | 1.53775 | 74.70 | S-FPM3.OHARA |
| 10 | −5.5901 | 0.1153 | | | |
| 11 (St) | ∞ | DD[11] | | | |
| 12 | 15.7424 | 0.8262 | 1.57501 | 41.50 | S-TIL27.OHARA |
| 13 | −1.2262 | 0.2500 | 2.00100 | 29.14 | S-LAH99.OHARA |
| 14 | −6.0252 | DD[14] | | | |
| 15 | −1.2049 | 0.2987 | 1.95906 | 17.47 | S-NPH3.OHARA |
| 16 | −1.6501 | 0.1000 | | | |
| 17 | 3.1572 | 0.2600 | 1.43875 | 94.66 | S-FPL55.OHARA |
| 18 | 2.9307 | 0.5096 | 1.85150 | 40.78 | S-LAH89.OHARA |
| 19 | −26.9795 | 0.3532 | | | |
| 20 | ∞ | 2.4804 | 1.84666 | 23.78 | S-TIH53W.OHARA |
| 21 | ∞ | 0.1400 | 1.51633 | 64.14 | S-BSL7.OHARA |
| 22 | ∞ | 0.0000 | | | |

TABLE 4
Example 2

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.90 | 1.90 |
| Zoom Ratio | 1.00 | 1.43 | 0.99 | 1.46 |
| Focal Length | 0.847 | 1.212 | 0.836 | 1.233 |
| F-Number | 4.16 | 5.19 | 4.19 | 5.38 |
| Maximum Full Angle | 163.0 | 87.6 | 152.2 | 84.2 |
| of View[°] | | | | |
| Maximum Image Height | 0.842 | 0.842 | 0.842 | 0.842 |
| DD[7] | 2.5568 | 1.2434 | 2.5568 | 1.2434 |
| DD[11] | 0.4809 | 0.1174 | 0.8884 | 0.5248 |
| DD[14] | 0.6286 | 2.3056 | 0.2211 | 1.8981 |

### Example 3

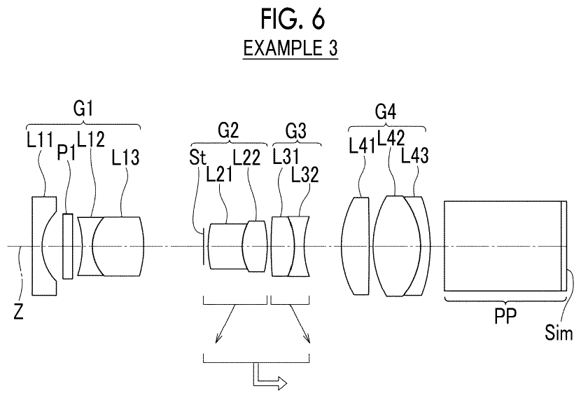

A cross-sectional view of a configuration of an endoscopic objective lens of Example 3 is shown in FIG. 6. The endoscopic objective lens of Example 3 consists of, in order from the object side to the image side, four lens groups including the first lens group G1 having negative optical power, the second lens group G2 having positive optical power, the third lens group G3 having negative optical power, and the fourth lens group G4 having positive optical power. consists of, in order from the object side to the image side, the aperture stop St, the lens L21, and the lens L22. The third lens group G3 consists of, in order from the object side to the image side, the lens L31 and the lens L32. The fourth lens group G4 consists of, in order from the object side to the image side, the lens L41, the lens L42, and the lens L43. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second moving lens group. The fourth lens group G4 corresponds to the second stationary lens group.

During changing the magnification from the wide angle end to the telephoto end, the second lens group G2 moves to the object side, the third lens group G3 moves to the image side, and other lens groups remain stationary with respect to the image plane Sim. During the focusing from the farthest point observation to the nearest point observation, the second lens group G2 and the third lens group G3 move to the image side in an integrated manner, and other lens groups remain stationary with respect to the image plane Sim.

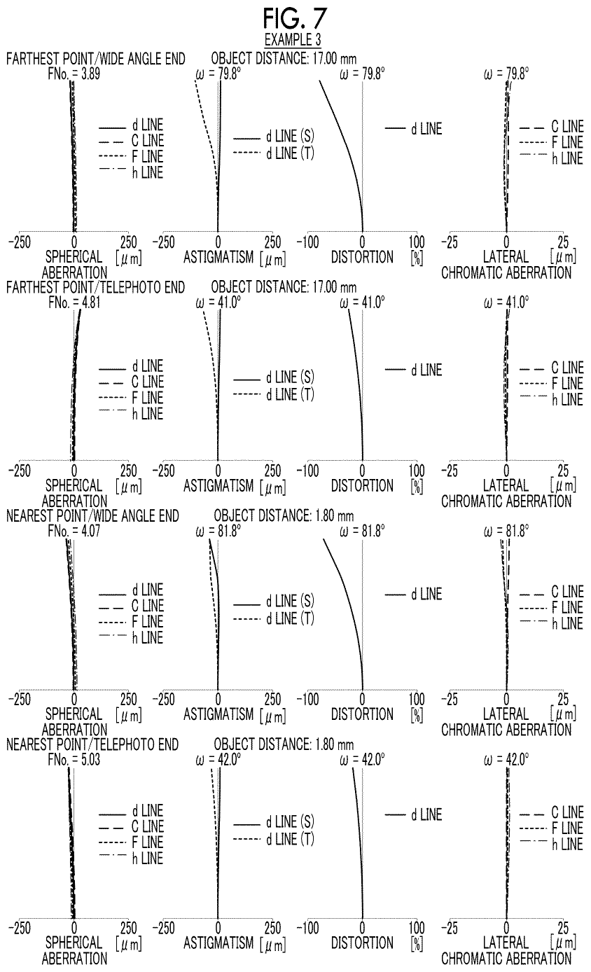

For the endoscopic objective lens of Example 3, Table 5 shows basic lens data, Table 6 shows specifications and variable surface spacings, and FIG. 7 shows each aberration diagram.

TABLE 5
Example 3

| Sn | R | D | Nd | νd | Material |
|---|---|---|---|---|---|
| 1 | −248.7427 | 0.2500 | 1.88300 | 40.76 | S-LAH58.OHARA |
| 2 | 1.2806 | 0.5378 | | | |
| 3 | ∞ | 0.2500 | 1.95906 | 17.47 | S-NPH3.OHARA |
| 4 | ∞ | 0.2511 | | | |
| 5 | −2.4139 | 0.2583 | 1.77250 | 49.60 | S-LAH66.OHARA |
| 6 | 1.1041 | 1.3267 | 1.63980 | 34.47 | S-TIM27.OHARA |
| 7 | −2.6652 | DD[7] | | | |
| 8 (St) | ∞ | 0.1173 | | | |
| 9 | 2.6500 | 0.8789 | 1.96300 | 24.11 | S-TIH57.OHARA |
| 10 | 1.3334 | 0.6452 | 1.65844 | 50.88 | S-BSM25.OHARA |
| 11 | −2.9747 | DD[11] | | | |
| 12 | 10.1774 | 0.5894 | 1.95906 | 17.47 | S-NPH3.OHARA |
| 13 | −1.9919 | 0.2500 | 1.96300 | 24.11 | S-TIH57.OHARA |
| 14 | 2.3147 | DD[14] | | | |
| 15 | 2.6066 | 0.7179 | 1.49700 | 81.54 | S-FPL51.OHARA |
| 16 | −45.9268 | 0.1000 | | | |
| 17 | 2.5799 | 1.2250 | 1.49700 | 81.54 | S-FPL51.OHARA |
| 18 | −2.0627 | 0.2500 | 2.00100 | 29.14 | S-LAH99.OHARA |
| 19 | −3.7566 | 0.3674 | | | |
| 20 | ∞ | 3.0120 | 1.84666 | 23.78 | S-TIH53W.OHARA |
| 21 | ∞ | 0.1400 | 1.51633 | 64.14 | S-BSL7.OHARA |
| 22 | ∞ | 0.0000 | | | |

TABLE 6
Example 3

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.48 | 0.94 | 1.33 |
| Focal Length | 0.886 | 1.308 | 0.828 | 1.177 |
| F-Number | 3.89 | 4.81 | 4.07 | 5.03 |
| Maximum Full Angle | 159.6 | 82.0 | 163.6 | 84.0 |
| of View[°] | | | | |
| Maximum Image Height | 0.837 | 0.837 | 0.837 | 0.837 |
| DD[7] | 1.5442 | 0.6187 | 2.0908 | 1.1653 |
| DD[11] | 0.1000 | 1.3386 | 0.1000 | 1.3386 |
| DD[14] | 0.9603 | 0.6472 | 0.4137 | 0.1006 |

### Example 4

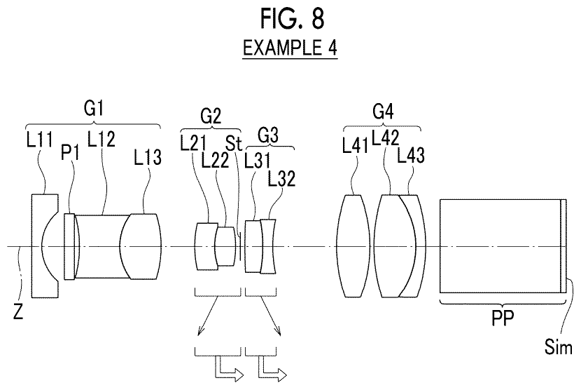

A cross-sectional view of a configuration of an endoscopic objective lens of Example 4 is shown in FIG. 8. The endoscopic objective lens of Example 4 consists of, in order from the object side to the image side, four lens groups including the first lens group G1 having negative optical power, the second lens group G2 having positive optical power, the third lens group G3 having negative optical power, and the fourth lens group G4 having positive optical power. consists of, in order from the object side to the image side, the lens L21, the lens L22, and the aperture stop St. The third lens group G3 consists of, in order from the object side to the image side, the lens L31 and the lens L32. The fourth lens group G4 consists of, in order from the object side to the image side, the lens L41, the lens L42, and the lens L43. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second moving lens group. The fourth lens group G4 corresponds to the second stationary lens group.

During changing the magnification from the wide angle end to the telephoto end, the second lens group G2 moves to the object side, the third lens group G3 moves to the image side, and other lens groups remain stationary with respect to the image plane Sim. During the focusing from the farthest point observation to the nearest point observation, the second lens group G2 and the third lens group G3 move to the image side along different trajectories from each other, and other lens groups remain stationary with respect to the image plane Sim.

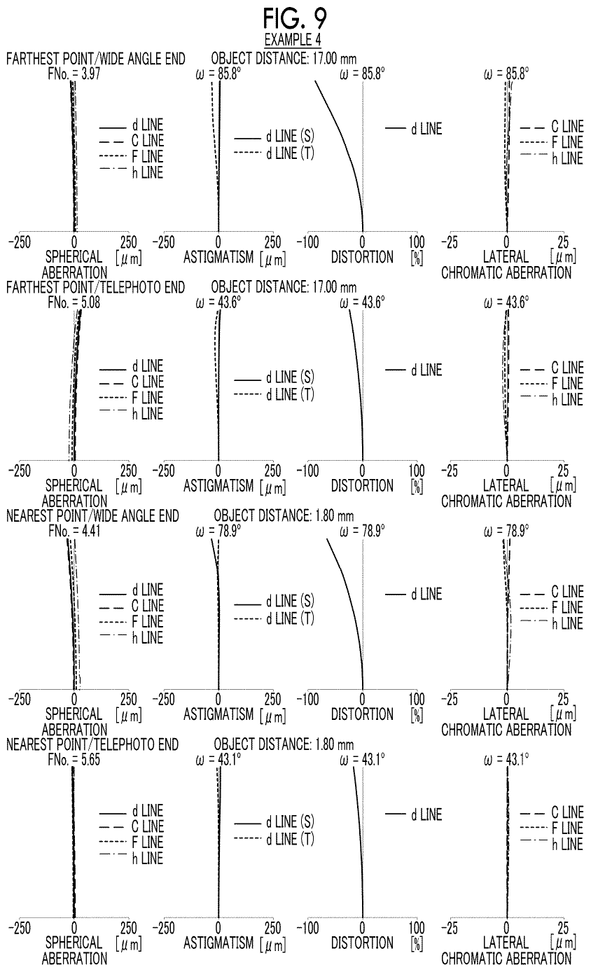

For the endoscopic objective lens of Example 4, Table 7 shows basic lens data, Table 8 shows specifications and variable surface spacings, and FIG. 9 shows each aberration diagram.

TABLE 7
Example 4

| Sn | R | D | Nd | νd | Material |
|---|---|---|---|---|---|
| 1 | ∞ | 0.2500 | 1.83481 | 42.74 | S-LAH55VS.OHARA |
| 2 | 1.1468 | 0.5712 | | | |
| 3 | ∞ | 0.2500 | 2.00100 | 29.14 | S-LAH99.OHARA |
| 4 | ∞ | 0.1281 | | | |
| 5 | −2.9444 | 1.0141 | 1.90525 | 35.04 | S-LAH93.OHARA |
| 6 | 1.3435 | 1.0414 | 1.76182 | 26.52 | S-TIH14.OHARA |
| 7 | −2.7606 | DD[7] | | | |
| 8 | 2.5826 | 0.5024 | 1.95906 | 17.47 | S-NPH3.OHARA |
| 9 | 1.5497 | 0.5272 | 1.51742 | 52.43 | S-NSL36.OHARA |
| 10 | −2.2660 | 0.1173 | | | |
| 11 (St) | ∞ | DD[11] | | | |
| 12 | 15.2957 | 0.4497 | 1.95906 | 17.47 | S-NPH3.OHARA |
| 13 | −3.5368 | 0.2500 | 2.00330 | 28.27 | S-LAH79.OHARA |
| 14 | 3.1748 | DD[14] | | | |
| 15 | 3.8481 | 0.8385 | 1.49700 | 81.54 | S-FPL51.OHARA |
| 16 | −3.6104 | 0.1000 | | | |
| 17 | 4.1619 | 1.0549 | 1.55200 | 70.70 | S-FPM5.OHARA |
| 18 | −2.0814 | 0.2500 | 2.00330 | 28.27 | S-LAH79.OHARA |
| 19 | −3.8493 | 0.3609 | | | |
| 20 | ∞ | 3.0353 | 1.84666 | 23.78 | S-TIH53W.OHARA |
| 21 | ∞ | 0.1400 | 1.51633 | 64.14 | S-BSL7.OHARA |
| 22 | ∞ | 0.0000 | | | |

TABLE 8
Example 4

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.45 | 0.99 | 1.38 |
| Focal Length | 0.829 | 1.199 | 0.818 | 1.147 |
| F-Number | 3.97 | 5.08 | 4.41 | 5.65 |
| Maximum Full Angle | 171.6 | 87.2 | 157.8 | 86.2 |
| of View[°] | | | | |
| Maximum Image Height | 0.847 | 0.847 | 0.847 | 0.847 |
| DD[7] | 0.8451 | 0.0863 | 1.5443 | 0.7855 |
| DD[11] | 0.1174 | 1.4697 | 0.3129 | 1.6653 |
| DD[14] | 1.5957 | 1.0021 | 0.7010 | 0.1074 |

### Example 5

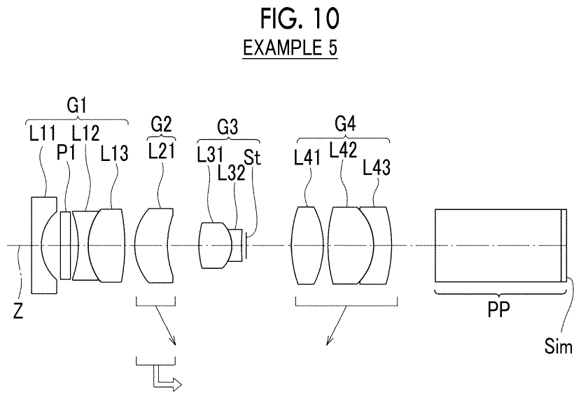

A cross-sectional view of a configuration of an endoscopic objective lens of Example 5 is shown in FIG. 10. The endoscopic objective lens of Example 5 consists of, in order from the object side to the image side, four lens groups including the first lens group G1 having negative optical power, the second lens group G2 having positive optical power, the third lens group G3 having negative optical power, and the fourth lens group G4 having positive optical power. consists of the lens L21. The third lens group G3 consists of, in order from the object side to the image side, the lens L31, the lens L32, and the aperture stop St. The fourth lens group G4 consists of, in order from the object side to the image side, the lens L41, the lens L42, and the lens L43. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second stationary lens group. The fourth lens group G4 corresponds to the second moving lens group.

During changing the magnification from the wide angle end to the telephoto end, the second lens group G2 moves to the image side, the fourth lens group G4 moves to the object side, and other lens groups remain stationary with respect to the image plane Sim. During the focusing from the farthest point observation to the nearest point observation, the second lens group G2 moves to the image side, and other lens groups remain stationary with respect to the image plane Sim.

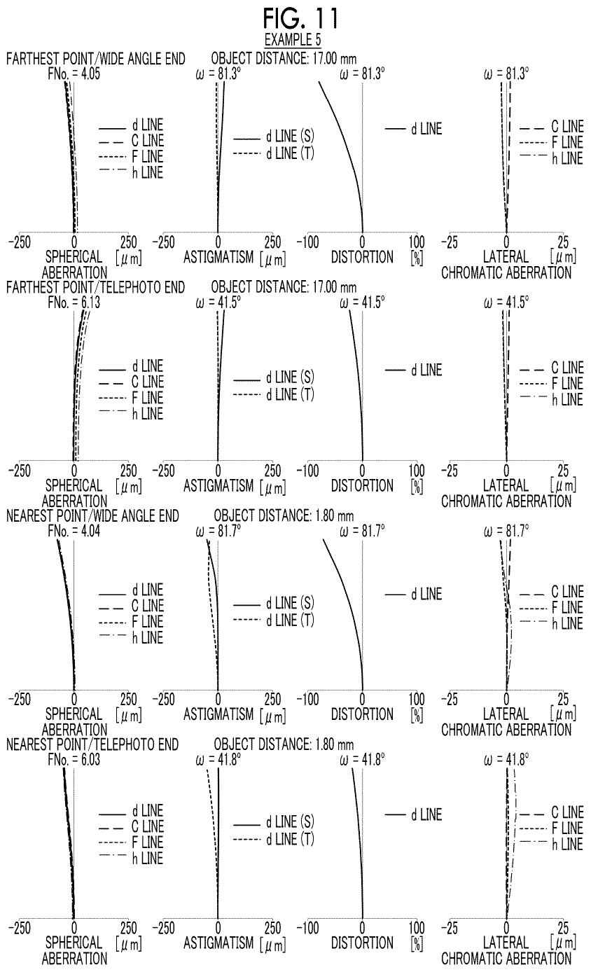

For the endoscopic objective lens of Example 5, Table 9 shows basic lens data, Table 10 shows specifications and variable surface spacings, and FIG. 11 shows each aberration diagram.

TABLE 9
Example 5

| Sn | R | D | Nd | νd | Material |
|---|---|---|---|---|---|
| 1 | ∞ | 0.2488 | 2.00100 | 29.14 | S-LAH99.OHARA |
| 2 | 1.1823 | 0.4891 | | | |
| 3 | ∞ | 0.2500 | 2.00100 | 29.14 | S-LAH99.OHARA |
| 4 | ∞ | 0.2054 | | | |
| 5 | −2.4996 | 0.2508 | 1.77250 | 49.60 | S-LAH66.OHARA |
| 6 | 1.5369 | 0.9251 | 1.72825 | 28.46 | S-TIH10.OHARA |
| 7 | −4.9528 | DD[7] | | | |
| 8 | 1.4322 | 0.8381 | 1.59522 | 67.73 | S-FPM2.OHARA |
| 9 | 2.2451 | DD[9] | | | |
| 10 | 2.1273 | 0.8409 | 1.63980 | 34.47 | S-TIM27.OHARA |
| 11 | −0.8314 | 0.2500 | 1.90525 | 35.04 | S-LAH93.OHARA |
| 12 | 5.8295 | 0.1176 | | | |
| 13 (St) | ∞ | DD[13] | | | |
| 14 | 2.9793 | 0.8132 | 1.49700 | 81.54 | S-FPL51.OHARA |
| 15 | −2.1611 | 0.1173 | | | |
| 16 | 3.7151 | 1.1696 | 1.49700 | 81.54 | S-FPL51.OHARA |
| 17 | −1.3568 | 0.4565 | 1.90525 | 35.04 | S-LAH93.OHARA |
| 18 | −3.5548 | DD[18] | | | |
| 19 | ∞ | 3.2000 | 1.88300 | 40.76 | S-LAH58.OHARA |
| 20 | ∞ | 0.1400 | 1.51633 | 64.14 | S-BSL7.OHARA |
| 21 | ∞ | 0.0000 | | | |

TABLE 10
Example 5

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.48 | 0.99 | 1.37 |
| Focal Length | 0.843 | 1.249 | 0.832 | 1.158 |
| F-Number | 4.05 | 6.13 | 4.04 | 6.03 |
| Maximum Full Angle | 162.6 | 83.0 | 163.4 | 83.6 |
| of View[°] | | | | |
| Maximum Image Height | 0.842 | 0.842 | 0.842 | 0.842 |
| DD[7] | 0.2505 | 0.4229 | 0.6227 | 0.7952 |
| DD[9] | 0.8019 | 0.6295 | 0.4297 | 0.2572 |
| DD[13] | 1.1430 | 0.2804 | 1.1430 | 0.2804 |
| DD[18] | 1.1175 | 1.9801 | 1.1175 | 1.9801 |

### Example 6

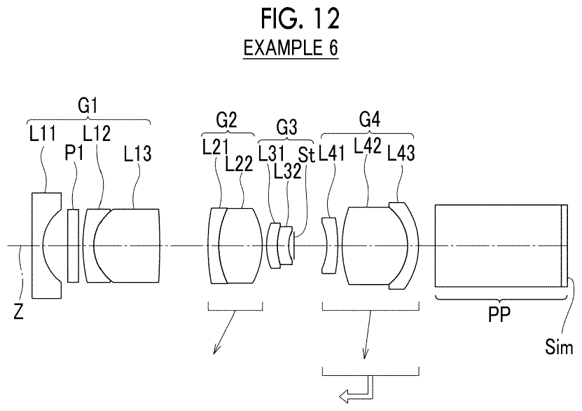

A cross-sectional view of a configuration of an endoscopic objective lens of Example 6 is shown in FIG. 12. The endoscopic objective lens of Example 6 consists of, in order from the object side to the image side, four lens groups including the first lens group G1 having negative optical power, the second lens group G2 having positive optical power, the third lens group G3 having negative optical power, and the fourth lens group G4 having positive optical power. consists of, in order from the object side to the image side, the lens L21 and the lens L22. The third lens group G3 consists of, in order from the object side to the image side, the lens L31, the lens L32, and the aperture stop St. The fourth lens group G4 consists of, in order from the object side to the image side, the lens L41, the lens L42, and the lens L43. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second stationary lens group. The fourth lens group G4 corresponds to the second moving lens group.

During changing the magnification from the wide angle end to the telephoto end, the second lens group G2 and the fourth lens group G4 move to the object side along different trajectories from each other, and other lens groups remain stationary with respect to the image plane Sim. During the focusing from the farthest point observation to the nearest point observation, the fourth lens group G4 moves to the object side, and other lens groups remain stationary with respect to the image plane Sim.

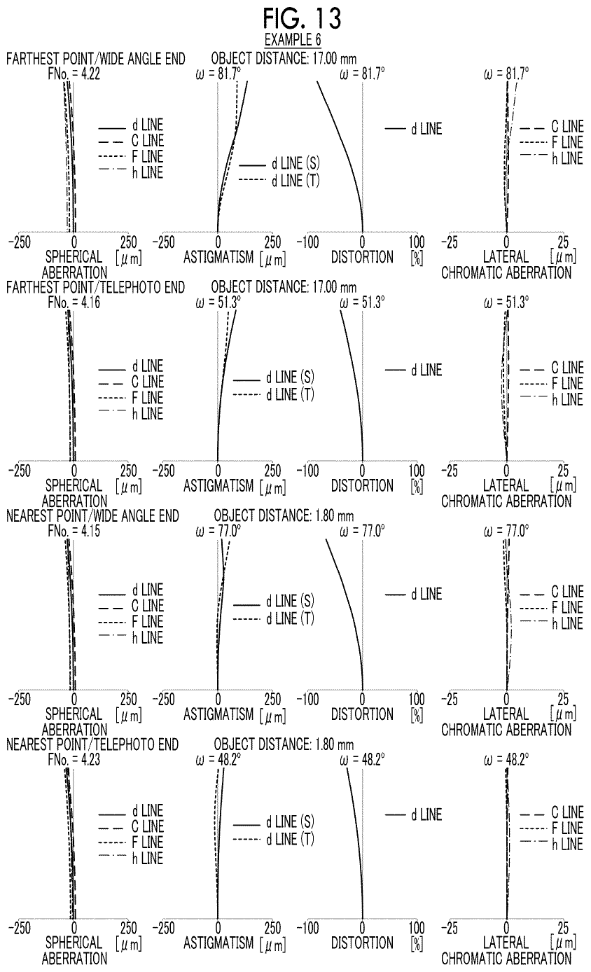

For the endoscopic objective lens of Example 6, Table 11 shows basic lens data, Table 12 shows specifications and variable surface spacings, and FIG. 13 shows each aberration diagram.

TABLE 11
Example 6

| Sn | R | D | Nd | νd | Material |
|---|---|---|---|---|---|
| 1 | ∞ | 0.2500 | 2.00100 | 29.14 | S-LAH99.OHARA |
| 2 | 1.0485 | 0.5731 | | | |
| 3 | ∞ | 0.2500 | 1.95906 | 17.47 | S-NPH3.OHARA |
| 4 | ∞ | 0.1000 | | | |
| 5 | 3.6885 | 0.2500 | 1.88300 | 40.76 | S-LAH58.OHARA |
| 6 | 1.0387 | 1.5082 | 1.72825 | 28.46 | S-TIH10.OHARA |
| 7 | −14.9363 | DD[7] | | | |
| 8 | 4.8897 | 0.2500 | 1.95906 | 17.47 | S-NPH3.OHARA |
| 9 | 1.9666 | 1.0004 | 1.60562 | 43.71 | S-BAM4.OHARA |
| 10 | −1.7893 | DD[10] | | | |
| 11 | 1.3972 | 0.2500 | 1.95906 | 17.47 | S-NPH3.OHARA |
| 12 | 1.5322 | 0.2600 | 1.54072 | 47.23 | S-TIL2.OHARA |
| 13 | 0.8844 | 0.1118 | | | |
| 14 (St) | ∞ | DD[14] | | | |
| 15 | −1.2899 | 0.2500 | 1.43875 | 94.66 | S-FPL55.OHARA |
| 16 | −3.6863 | 0.1000 | | | |
| 17 | 2.6121 | 1.5100 | 1.59522 | 67.73 | S-FPM2.OHARA |
| 18 | −1.1392 | 0.2500 | 1.95906 | 17.47 | S-NPH3.OHARA |
| 19 | −1.6863 | DD[19] | | | |
| 20 | ∞ | 2.8964 | 1.88300 | 40.76 | S-LAH58.OHARA |
| 21 | ∞ | 0.1400 | 1.51633 | 64.14 | S-BSL7.OHARA |
| 22 | ∞ | 0.0000 | | | |

TABLE 12
Example 6

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.19 | 0.95 | 1.14 |
| Focal Length | 0.970 | 1.157 | 0.919 | 1.101 |
| F-Number | 4.22 | 4.16 | 4.15 | 4.23 |
| Maximum Full Angle | 163.4 | 102.6 | 154.0 | 96.4 |
| of View[°] | | | | |
| Maximum Image Height | 0.842 | 0.842 | 0.842 | 0.842 |
| DD[7] | 1.1111 | 0.5308 | 1.1111 | 0.5308 |
| DD[10] | 0.1000 | 0.6803 | 0.1000 | 0.6803 |
| DD[14] | 0.7583 | 0.5558 | 0.3805 | 0.1779 |
| DD[19] | 0.3829 | 0.5854 | 0.7607 | 0.9633 |

### Example 7

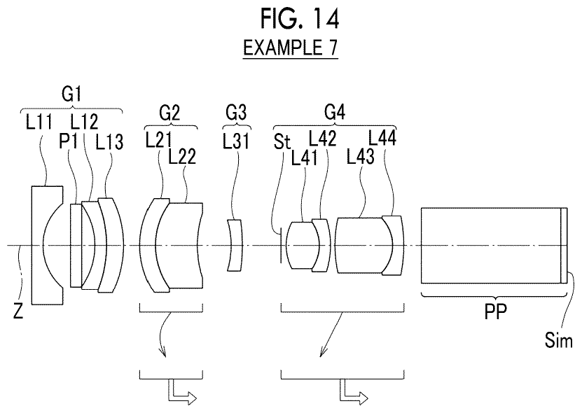

A cross-sectional view of a configuration of an endoscopic objective lens of Example 7 is shown in FIG. 14. The endoscopic objective lens of Example 7 consists of, in order from the object side to the image side, four lens groups including the first lens group G1 having negative optical power, the second lens group G2 having positive optical power, the third lens group G3 having negative optical power, and the fourth lens group G4 having positive optical power. The first lens group G1 consists of, in order from the object side to the image side, the consists of, in order from the object side to the image side, the lens L21 and the lens L22. The third lens group G3 consists of the lens L31. The fourth lens group G4 consists of, in order from the object side to the image side, the aperture stop St, the lens L41, the lens L42, the lens L43, and a lens L44. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second stationary lens group. The fourth lens group G4 corresponds to the second moving lens group.

During changing the magnification from the wide angle end to the telephoto end, the second lens group G2 moves to the object side and then moves to the image side, the fourth lens group G4 moves to the object side, and other lens groups remain stationary with respect to the image plane Sim. During the focusing from the farthest point observation to the nearest point observation, the second lens group G2 and the fourth lens group G4 move to the image side along different trajectories from each other, and other lens groups remain stationary with respect to the image plane Sim.

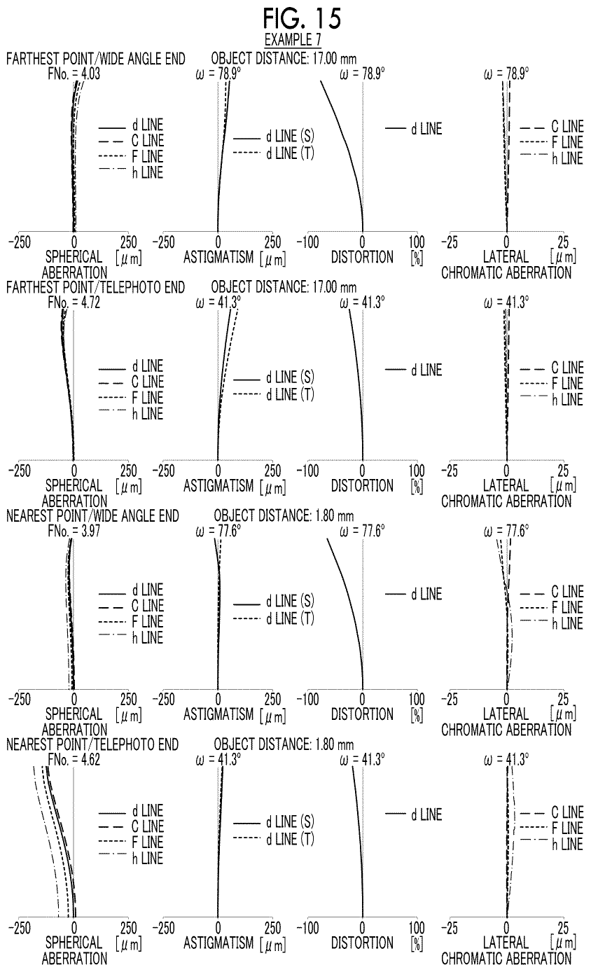

For the endoscopic objective lens of Example 7, Table 13 shows basic lens data, Table 14 shows specifications and variable surface spacings, and FIG. 15 shows each aberration diagram.

TABLE 13
Example 7

| Sn | R | D | Nd | νd | Material |
|---|---|---|---|---|---|
| 1 | ∞ | 0.2500 | 2.00100 | 29.14 | S-LAH99.OHARA |
| 2 | 1.2430 | 0.6151 | | | |
| 3 | ∞ | 0.2500 | 2.00100 | 29.14 | S-LAH99.OHARA |
| 4 | ∞ | 0.2969 | | | |
| 5 | −1.5976 | 0.2600 | 1.59522 | 67.73 | S-FPM2.OHARA |
| 6 | −2.7248 | 0.3820 | 1.89286 | 20.36 | S-NPH4.OHARA |
| 7 | −2.3823 | DD[7] | | | |
| 8 | 1.9421 | 0.3441 | 1.95906 | 17.47 | S-NPH3.OHARA |
| 9 | 1.6304 | 0.9361 | 1.96300 | 24.11 | S-TIH57.OHARA |
| 10 | 2.3668 | DD[10] | | | |
| 11 | −1.9644 | 0.2500 | 1.59522 | 67.73 | S-FPM2.OHARA |
| 12 | −4.3909 | DD[12] | | | |
| 13 (St) | ∞ | 0.1163 | | | |
| 14 | 1.1313 | 0.7418 | 1.43875 | 94.66 | S-FPL55.OHARA |
| 15 | −0.9240 | 0.2500 | 1.83481 | 42.74 | S-LAH55VS.OHARA |
| 16 | −1.9701 | 0.1000 | | | |
| 17 | 2.8668 | 1.2897 | 1.63980 | 34.47 | S-TIM27.OHARA |
| 18 | −0.8936 | 0.2500 | 2.00330 | 28.27 | S-LAH79.OHARA |
| 19 | −3.3214 | DD[19] | | | |
| 20 | ∞ | 3.1115 | 1.95375 | 32.32 | S-LAH98.OHARA |
| 21 | ∞ | 0.1400 | 1.51633 | 64.14 | S-BSL7.OHARA |
| 22 | ∞ | 0.0000 | | | |

TABLE 14
Example 7

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.43 | 0.95 | 1.29 |
| Focal Length | 0.883 | 1.260 | 0.835 | 1.137 |
| F-Number | 4.03 | 4.72 | 3.97 | 4.62 |
| Maximum Full Angle | 157.8 | 82.6 | 155.2 | 82.6 |
| of View[°] | | | | |
| Maximum Image Height | 0.837 | 0.837 | 0.837 | 0.837 |
| DD[7] | 0.3584 | 0.1786 | 0.9494 | 0.7696 |
| DD[10] | 0.7567 | 0.9365 | 0.1657 | 0.3455 |
| DD[12] | 0.8644 | 0.1048 | 0.8897 | 0.1301 |
| DD[19] | 0.3918 | 1.1514 | 0.3665 | 1.1261 |

### Example 8

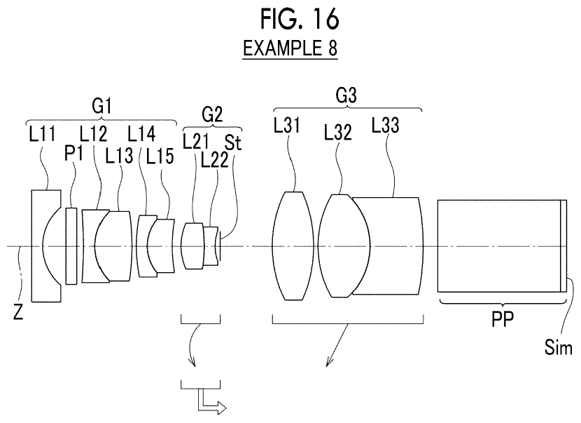

A cross-sectional view of a configuration of an endoscopic objective lens of Example 8 is shown in FIG. 16. The endoscopic objective lens of Example 8 consists of, in order from the object side to the image side, three lens groups including the first lens group G1 having negative optical power, the second lens group G2 having positive optical power, and the third lens group G3 having positive optical power.

The first lens group G1 consists of, in order from the object side to the image side, the lens L11, the optical member P1, the lens L12, the lens L13, a lens L14, and a lens L15. The second lens group G2 consists of, in order from the object side to the image side, the lens L21, the lens L22, and the aperture stop St. The third lens group G3 consists of, in order from the object side to the image side, the lens L31, the lens L32, and a lens L33. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second moving lens group.

During changing the magnification from the wide angle end to the telephoto end, the second lens group G2 moves to the object side and then moves to the image side, the third lens group G3 moves to the object side, and other lens groups remain stationary with respect to the image plane Sim. During the focusing from the farthest point observation to the nearest point observation, the second lens group G2 moves to the image side, and other lens groups remain stationary with respect to the image plane Sim.

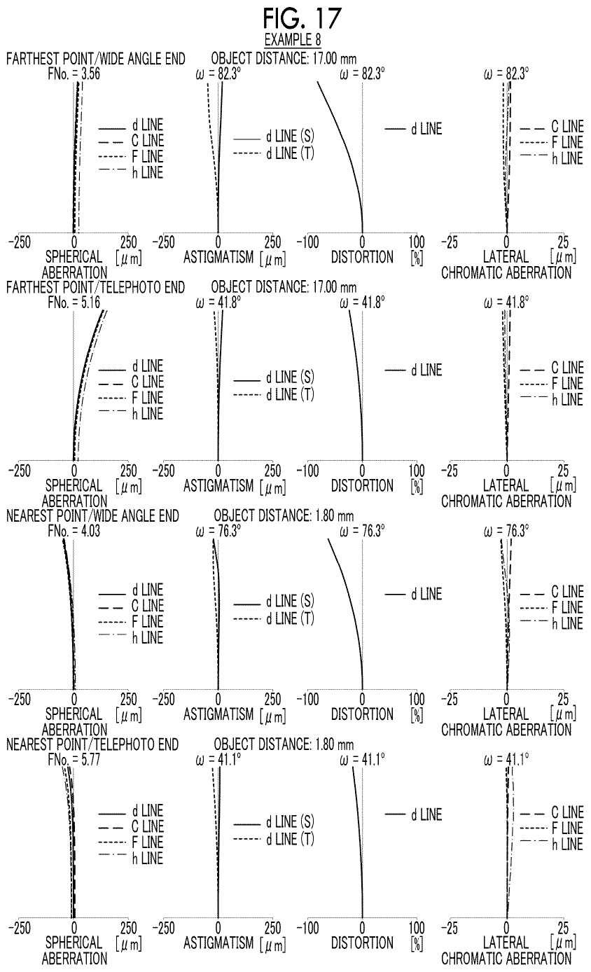

For the endoscopic objective lens of Example 8, Table 15 shows basic lens data, Table 16 shows specifications and variable surface spacings, and FIG. 17 shows each aberration diagram.

TABLE 15
Example 8

| Sn | R | D | Nd | νd | Material |
|---|---|---|---|---|---|
| 1 | ∞ | 0.2500 | 1.95375 | 32.32 | S-LAH98.OHARA |
| 2 | 1.1730 | 0.5340 | | | |
| 3 | ∞ | 0.2500 | 2.00100 | 29.14 | S-LAH99.OHARA |
| 4 | ∞ | 0.1500 | | | |
| 5 | −10.8064 | 0.2600 | 1.75500 | 52.32 | S-LAH97.OHARA |
| 6 | 1.1401 | 0.8425 | 1.80518 | 25.42 | S-TIH6.OHARA |
| 7 | −5.3725 | 0.1000 | | | |
| 8 | 3.5979 | 0.2500 | 2.00100 | 29.14 | S-LAH99.OHARA |
| 9 | 0.9508 | 0.5719 | 1.63980 | 34.47 | S-TIM27.OHARA |
| 10 | 3.4340 | DD[10] | | | |
| 11 | 1.3057 | 0.5321 | 1.59522 | 67.73 | S-FPM2.OHARA |
| 12 | −3.4655 | 0.2500 | 1.43875 | 94.66 | S-FPL55.OHARA |
| 13 | 1.1138 | 0.1152 | | | |
| 14 (St) | ∞ | DD[14] | | | |
| 15 | 2.8397 | 0.9356 | 1.59522 | 67.73 | S-FPM2.OHARA |
| 16 | −3.1075 | 0.1000 | | | |
| 17 | 2.3815 | 1.1930 | 1.53775 | 74.70 | S-FPM3.OHARA |
| 18 | −1.6234 | 1.2161 | 2.00100 | 29.14 | S-LAH99.OHARA |
| 19 | −5.7580 | DD[19] | | | |
| 20 | ∞ | 2.8002 | 1.84666 | 23.78 | S-TIH53W.OHARA |
| 21 | ∞ | 0.1400 | 1.51633 | 64.14 | S-BSL7.OHARA |
| 22 | ∞ | 0.0000 | | | |

TABLE 16
Example 8

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.43 | 0.97 | 1.32 |
| Focal Length | 0.866 | 1.242 | 0.839 | 1.146 |
| F-Number | 3.56 | 5.16 | 4.03 | 5.7 |
| Maximum Full Angle | 164.6 | 83.6 | 152.6 | 82.2 |
| of View[°] | | | | |
| Maximum Image Height | 0.842 | 0.842 | 0.842 | 0.842 |
| DD[10] | 0.2015 | 0.1301 | 0.6183 | 0.5470 |
| DD[14] | 1.1940 | 0.4675 | 0.7772 | 0.0506 |
| DD[19] | 0.3362 | 1.1341 | 0.3362 | 1.1341 |

### Example 9

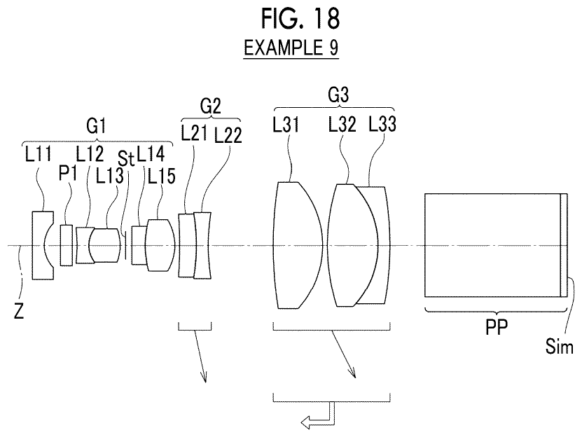

A cross-sectional view of a configuration of an endoscopic objective lens of Example 9 is shown in FIG. 18. The endoscopic objective lens of Example 9 consists of, in order from the object side to the image side, three lens groups including the first lens group G1 having positive optical power, the second lens group G2 having negative optical power, and the third lens group G3 having positive optical power.

The first lens group G1 consists of, in order from the object side to the image side, the lens L11, the optical member P1, the lens L12, the lens L13, the aperture stop St, the lens L14, and the lens L15. The second lens group G2 consists of, in order from the object side to the image side, the lens L21 and the lens L22. The third lens group G3 consists of, in order from the object side to the image side, the lens L31, the lens L32, and the lens L33. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second moving lens group.

During changing the magnification from the wide angle end to the telephoto end, the second lens group G2 and the third lens group G3 move to the image side along different trajectories from each other, and other lens groups remain stationary with respect to the image plane Sim. During the focusing from the farthest point observation to the nearest point observation, the third lens group G3 moves to the object side, and other lens groups remain stationary with respect to the image plane Sim.

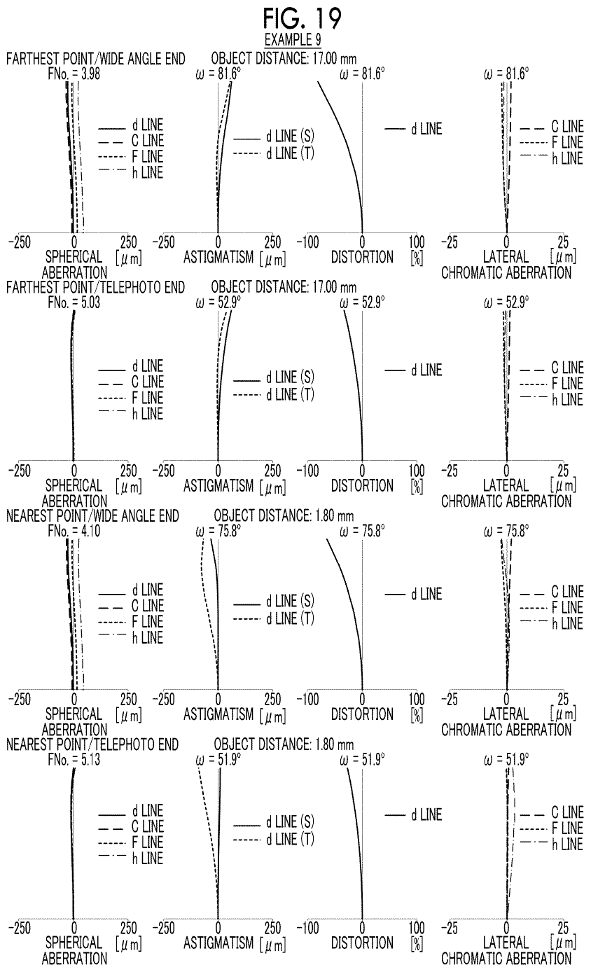

For the endoscopic objective lens of Example 9, Table 17 shows basic lens data, Table 18 shows specifications and variable surface spacings, and FIG. 19 shows each aberration diagram.

TABLE 17
Example 9

| Sn | R | D | Nd | νd | Material |
|---|---|---|---|---|---|
| 1 | ∞ | 0.2500 | 2.00100 | 29.14 | S-LAH99.OHARA |
| 2 | 0.6751 | 0.3382 | | | |
| 3 | ∞ | 0.2500 | 1.95906 | 17.47 | S-NPH3.OHARA |
| 4 | ∞ | 0.1000 | | | |
| 5 | −3.6229 | 0.2500 | 2.00100 | 29.14 | S-LAH99.OHARA |
| 6 | 0.5874 | 0.6485 | 1.80518 | 25.42 | S-TIH6.OHARA |
| 7 | −1.0408 | 0.1160 | | | |
| 8 (St) | ∞ | 0.1311 | | | |
| 9 | −44.9100 | 0.2819 | 2.00100 | 29.14 | S-LAH99.OHARA |
| 10 | 2.1214 | 0.6227 | 1.53775 | 74.70 | S-FPM3.OHARA |
| 11 | −1.0593 | DD[11] | | | |
| 12 | −11.4334 | 0.3542 | 1.95906 | 17.47 | S-NPH3.OHARA |
| 13 | −4.1451 | 0.2500 | 2.00100 | 29.14 | S-LAH99.OHARA |
| 14 | 4.3914 | DD[14] | | | |
| 15 | 8.4789 | 1.0308 | 1.43875 | 94.66 | S-FPL55.OHARA |
| 16 | −2.0025 | 0.1000 | | | |
| 17 | 4.3094 | 1.0673 | 1.69680 | 55.53 | S-LAL14.OHARA |
| 18 | −1.8699 | 0.2500 | 2.00100 | 29.14 | S-LAH99.OHARA |
| 19 | −6.8594 | DD[19] | | | |
| 20 | ∞ | 2.8448 | 1.88300 | 40.76 | S-LAH58.OHARA |
| 21 | ∞ | 0.1400 | 1.51633 | 64.14 | S-BSL7.OHARA |
| 22 | ∞ | 0.0000 | | | |

TABLE 18
Example 9

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.26 | 1.04 | 1.28 |
| Focal Length | 0.771 | 0.973 | 0.798 | 0.989 |
| F-Number | 3.98 | 5.03 | 4.10 | 5.13 |
| Maximum Full Angle | 163.2 | 105.8 | 151.6 | 103.8 |
| of View[°] | | | | |
| Maximum Image Height | 0.842 | 0.842 | 0.842 | 0.842 |
| DD[11] | 0.1000 | 0.9977 | 0.1000 | 0.9977 |
| DD[14] | 1.3676 | 0.7956 | 1.0691 | 0.4971 |
| DD[19] | 0.7385 | 0.4127 | 1.0370 | 0.7113 |

### Example 10

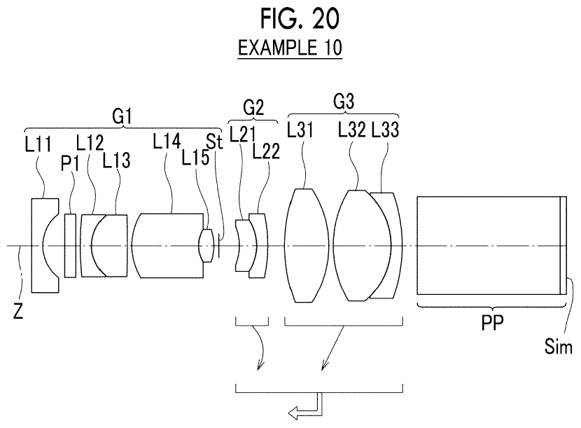

A cross-sectional view of a configuration of an endoscopic objective lens of Example 10 is shown in FIG. 20. The endoscopic objective lens of Example 10 consists of, in order from the object side to the image side, three lens groups including the first lens group G1 having positive optical power, the second lens group G2 having negative optical power, and the third lens group G3 having positive optical power.

The first lens group G1 consists of, in order from the object side to the image side, the lens L11, the optical member P1, the lens L12, the lens L13, the lens L14, the lens L15, and the aperture stop St. The second lens group G2 consists of, in order from the object side to the image side, the lens L21 and the lens L22. The third lens group G3 consists of, in order from the object side to the image side, the lens L31, the lens L32, and the lens L33. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second moving lens group.

During changing the magnification from the wide angle end to the telephoto end, the second lens group G2 moves to the image side and then moves to the object side, the third lens group G3 moves to the object side, and other lens groups remain stationary with respect to the image plane Sim. During the focusing from the farthest point observation to the nearest point observation, the second lens group G2 and the third lens group G3 move to the object side in an integrated manner, and other lens groups remain stationary with respect to the image plane Sim.

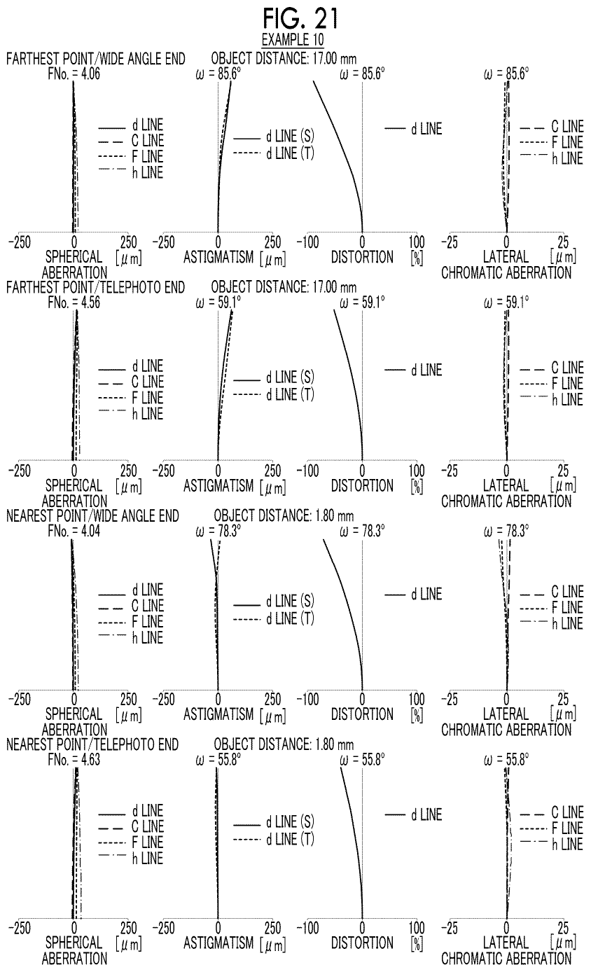

For the endoscopic objective lens of Example 10, Table 19 shows basic lens data, Table 20 shows specifications and variable surface spacings, and FIG. 21 shows each aberration diagram.

TABLE 19
Example 10

| Sn | R | D | Nd | νd | Material |
|---|---|---|---|---|---|
| 1 | ∞ | 0.2500 | 2.00100 | 29.14 | S-LAH99.OHARA |
| 2 | 0.9075 | 0.4919 | | | |
| 3 | ∞ | 0.2500 | 2.00100 | 29.14 | S-LAH99.OHARA |
| 4 | ∞ | 0.1000 | | | |
| 5 | 6.6821 | 0.2500 | 1.88300 | 40.76 | S-LAH58.OHARA |
| 6 | 0.8809 | 0.7969 | 1.67270 | 32.10 | S-TIM25.OHARA |
| 7 | −27.7886 | 0.1000 | | | |
| 8 | 1.3803 | 1.5000 | 2.00100 | 29.14 | S-LAH99.OHARA |
| 9 | 0.7143 | 0.3432 | 1.51742 | 52.43 | S-NSL36.OHARA |
| 10 | −1.1436 | 0.1086 | | | |
| 11 (St) | ∞ | DD[11] | | | |
| 12 | −1.1800 | 0.3881 | 1.95906 | 17.47 | S-NPH3.OHARA |
| 13 | −1.0751 | 0.2500 | 2.00100 | 29.14 | S-LAH99.OHARA |
| 14 | −3.4908 | DD[14] | | | |
| 15 | 3.6558 | 0.9841 | 1.59522 | 67.73 | S-FPM2.OHARA |
| 16 | −2.3501 | 0.1000 | | | |
| 17 | 2.4386 | 1.2981 | 1.49700 | 81.54 | S-FPL51.OHARA |
| 18 | −1.6242 | 0.2500 | 2.00100 | 29.14 | S-LAH99.OHARA |
| 19 | −3.5390 | DD[19] | | | |
| 20 | ∞ | 3.2000 | 1.88300 | 40.76 | S-LAH58.OHARA |
| 21 | ∞ | 0.1400 | 1.51633 | 64.14 | S-BSL7.OHARA |
| 22 | ∞ | 0.0000 | | | |

TABLE 20
Example 10

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.11 | 0.96 | 1.08 |
| Focal Length | 0.965 | 1.075 | 0.930 | 1.037 |
| F-Number | 4.06 | 4.56 | 4.04 | 4.63 |
| Maximum Full Angle | 171.2 | 118.2 | 156.6 | 111.6 |
| of View[°] | | | | |
| Maximum Image Height | 0.842 | 0.842 | 0.842 | 0.842 |
| DD[11] | 0.4638 | 0.4868 | 0.1264 | 0.1494 |
| DD[14] | 0.3761 | 0.1470 | 0.3761 | 0.1470 |
| DD[19] | 0.3372 | 0.5432 | 0.6746 | 0.8807 |

### Example 11

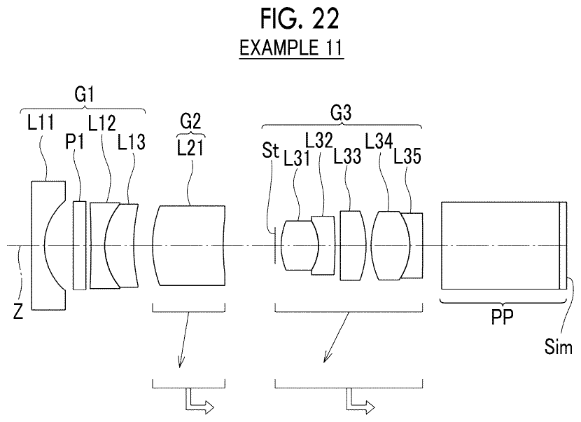

A cross-sectional view of a configuration of an endoscopic objective lens of Example 11 is shown in FIG. 22. The endoscopic objective lens of Example 11 consists of, in order from the object side to the image side, three lens groups including the first lens group G1 having negative optical power, the second lens group G2 having positive optical power, and the third lens group G3 having positive optical power.

The first lens group G1 consists of, in order from the object side to the image side, the lens L11, the optical member P1, the lens L12, and the lens L13. The second lens group G2 consists of the lens L21. The third lens group G3 consists of, in order from the object side to the image side, the aperture stop St, the lens L31, the lens L32, the lens L33, a lens L34, and a lens L35. The optical member P1 is assumed to be a filter and the like and is a member that has an incident surface and an emission surface parallel to each other, and does not have optical power.

During changing the magnification from the wide angle end to the telephoto end, the second lens group G2 and the third lens group G3 move to the object side along different trajectories from each other, and other lens groups remain stationary with respect to the image plane Sim. During the focusing from the farthest point observation to the nearest point observation, the second lens group G2 and the third lens group G3 move to the image side along different trajectories from each other, and other lens groups remain stationary with respect to the image plane Sim.

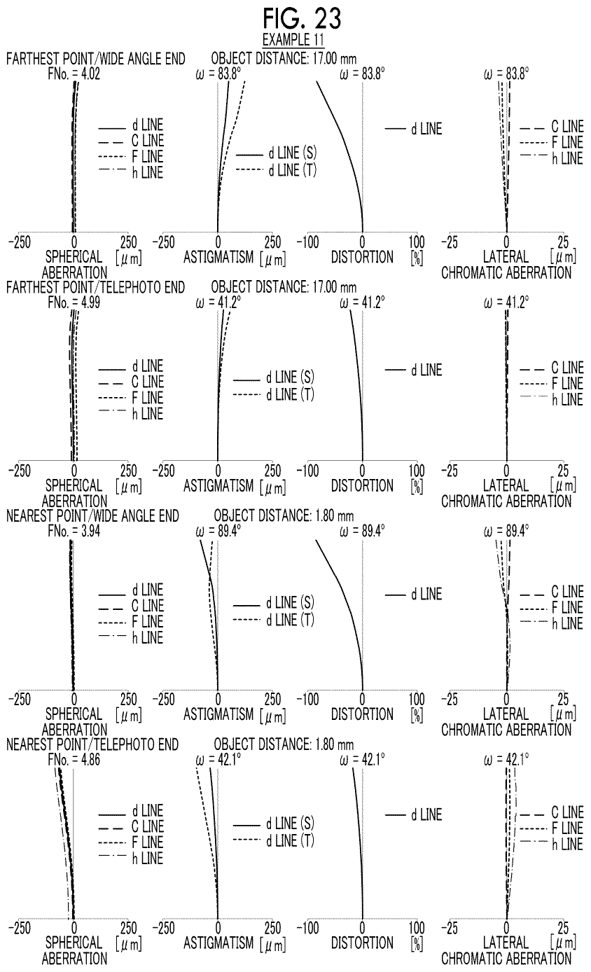

For the endoscopic objective lens of Example 11, Table 21 shows basic lens data, Table 22 shows specifications and variable surface spacings, and FIG. 23 shows each aberration diagram.

TABLE 21
Example 11

| Sn | R | D | Nd | νd | Material |
|---|---|---|---|---|---|
| 1 | ∞ | 0.2500 | 2.00100 | 29.14 | S-LAH99.OHARA |
| 2 | 1.1727 | 0.5671 | | | |
| 3 | ∞ | 0.2500 | 2.00100 | 29.14 | S-LAH99.OHARA |
| 4 | ∞ | 0.1000 | | | |
| 5 | −19.1252 | 0.2600 | 1.75500 | 52.32 | S-LAH97.OHARA |
| 6 | 1.2821 | 0.5688 | 1.89286 | 20.36 | S-NPH4.OHARA |
| 7 | 3.6357 | DD[7] | | | |
| 8 | 2.2351 | 1.3882 | 1.85150 | 40.78 | S-LAH89.OHARA |
| 9 | 4.7484 | DD[9] | | | |
| 10 (St) | ∞ | 0.1175 | | | |
| 11 | 1.2765 | 0.7245 | 1.53775 | 74.70 | S-FPM3.OHARA |
| 12 | −0.9332 | 0.3248 | 1.96300 | 24.11 | S-TIH57.OHARA |
| 13 | −7.8439 | 0.1065 | | | |
| 14 | −76.2046 | 0.5046 | 1.95906 | 17.47 | S-NPH3.OHARA |
| 15 | −1.9867 | 0.1000 | | | |
| 16 | 1.6288 | 0.7618 | 1.43875 | 94.66 | S-FPL55.OHARA |
| 17 | −1.1930 | 0.2500 | 2.00330 | 28.27 | S-LAH79.OHARA |
| 18 | −75.0901 | DD[18] | | | |
| 19 | ∞ | 2.3080 | 1.88300 | 40.76 | S-LAH58.OHARA |
| 20 | ∞ | 0.1400 | 1.51633 | 64.14 | S-BSL7.OHARA |
| 21 | ∞ | 0.0000 | | | |

TABLE 22
Example 11

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.50 | 0.93 | 1.32 |
| Focal Length | 0.824 | 1.236 | 0.767 | 1.090 |
| F-Number | 4.02 | 4.99 | 3.94 | 4.86 |
| Maximum Full Angle | 167.6 | 82.4 | 178.8 | 84.2 |
| of View[°] | | | | |
| Maximum Image Height | 0.844 | 0.844 | 0.844 | 0.844 |
| DD[7] | 0.3719 | 0.0996 | 0.7199 | 0.4476 |
| DD[9] | 1.0223 | 0.4647 | 0.7218 | 0.1642 |
| DD[18] | 0.3749 | 1.2048 | 0.3275 | 1.1574 |

### Example 12

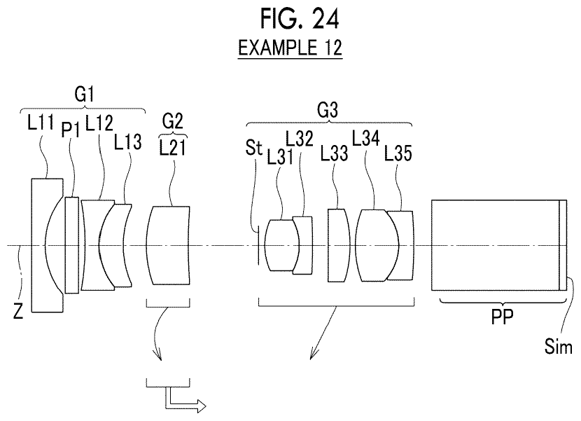

A cross-sectional view of a configuration of an endoscopic objective lens of Example 12 is shown in FIG. 24. The endoscopic objective lens of Example 12 consists of, in order from the object side to the image side, three lens groups including the first lens group G1 having negative optical power, the second lens group G2 having positive optical power, and the third lens group G3 having positive optical power.

The first lens group G1 consists of, in order from the object side to the image side, the lens L11, the optical member P1, the lens L12, and the lens L13. The second lens group G2 consists of the lens L21. The third lens group G3 consists of, in order from the object side to the image side, the aperture stop St, the lens L31, the lens L32, the lens L33, the lens L34, and the lens L35. The optical member P1 is assumed to be a filter and the like and is a member that has an incident surface and an emission surface parallel to each other, and does not have optical power.

During changing the magnification from the wide angle end to the telephoto end, the second lens group G2 moves to the object side and then moves to the image side, the third lens group G3 moves to the object side, and other lens groups remain stationary with respect to the image plane Sim. During the focusing from the farthest point observation to the nearest point observation, the second lens group G2 moves to the image side, and other lens groups remain stationary with respect to the image plane Sim.

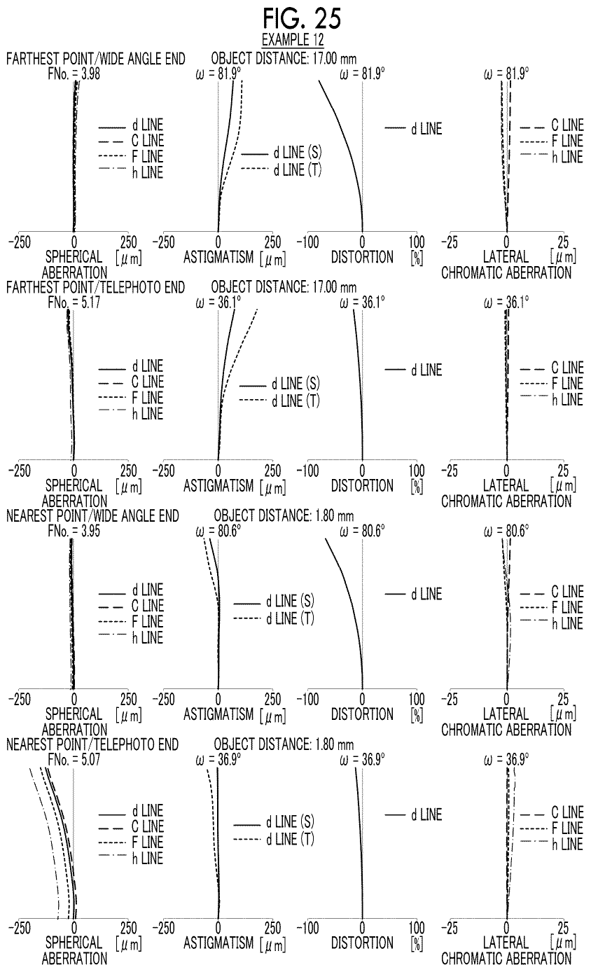

For the endoscopic objective lens of Example 12, Table 23 shows basic lens data, Table 24 shows specifications and variable surface spacings, Table 25 shows aspherical coefficients, and FIG. 25 shows each aberration diagram.

In the basic lens data, the surface number of an aspherical surface is marked with *, and a field of the curvature radius of the aspherical surface shows a numerical value of a paraxial curvature radius. In Table 25, a column of Sn shows the surface number of the aspherical surface, and columns of KA and Am show numerical values of the aspherical coefficients for each aspherical surface. Here, m in Am is an integer greater than or equal to 3 and varies depending on the surface. For example, for a fourteenth surface of Example 12, m=3, 4, 5, 6, . . . 12 is established. In the numerical values of the aspherical coefficients in Table 25, “En” (n: integer) means “×10+n”. KA and Am are aspherical coefficients in an aspheric equation represented by the following expression.

Zd=C×h²/{1+(1−KA×C²×h²)^{1/2}}+ΣAm×h^{m}
where
Zd: a depth of the aspherical surface (a length of a perpendicular line drawn from a point on the aspherical surface having a height h to a plane that is in contact with an aspherical surface apex and perpendicular to the optical axis Z),
h: a height (a distance from the optical axis Z to the lens surface),
C: a reciprocal of the paraxial curvature radius,
KA and Am: aspherical coefficients, and
Σ in the aspheric equation means a sum total related to m.

The same description method related to the aspherical surface applies to the examples described later.

TABLE 23
Example 12

| Sn | R | D | Nd | νd | Material |
|---|---|---|---|---|---|
| 1 | ∞ | 0.2544 | 2.00100 | 29.14 | S-LAH99.OHARA |
| 2 | 1.4684 | 0.3762 | | | |
| 3 | ∞ | 0.2500 | 2.00100 | 29.14 | S-LAH99.OHARA |
| 4 | ∞ | 0.1250 | | | |
| 5 | −4.9532 | 0.2600 | 1.71300 | 53.87 | S-LAL8.OHARA |
| 6 | 1.2241 | 0.4480 | 1.92286 | 18.90 | S-NPH2.OHARA |
| 7 | 1.7701 | DD[7] | | | |
| 8 | 2.1699 | 0.7891 | 1.84666 | 23.78 | S-TIH53W.OHARA |
| 9 | 9.4832 | DD[9] | | | |
| 10 (St) | ∞ | 0.1175 | | | |
| 11 | 1.1719 | 0.6402 | 1.51742 | 52.43 | S-NSL36.OHARA |
| 12 | −1.0140 | 0.2500 | 1.96300 | 24.11 | S-TIH57.OHARA |
| 13 | −10.7358 | 0.2932 | | | |
| *14 | 77.1083 | 0.4121 | 2.00178 | 19.32 | M-FDS2.HOYA |
| *15 | −2.3082 | 0.1000 | | | |
| 16 | 1.9513 | 0.8161 | 1.49700 | 81.54 | S-FPL51.OHARA |
| 17 | −0.9684 | 0.2833 | 2.00330 | 28.27 | S-LAH79.OHARA |
| 18 | −7.6472 | DD[18] | | | |
| 19 | ∞ | 2.3880 | 1.78472 | 25.68 | S-TIH11.OHARA |
| 20 | ∞ | 0.1400 | 1.51633 | 64.14 | S-BSL7.OHARA |
| 21 | ∞ | 0.0000 | | | |

TABLE 24
Example 12

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.73 | 0.96 | 1.52 |
| Focal Length | 0.787 | 1.363 | 0.752 | 1.198 |
| F-Number | 3.98 | 5.17 | 3.95 | 5.07 |
| Maximum Full Angle | 163.8 | 72.2 | 161.2 | 73.8 |
| of View[°] | | | | |
| Maximum Image Height | 0.842 | 0.842 | 0.842 | 0.842 |
| DD[7] | 0.4344 | 0.3221 | 0.6212 | 0.5089 |
| DD[9] | 1.3214 | 0.3077 | 1.1346 | 0.1209 |
| DD[18] | 0.3387 | 1.4647 | 0.3387 | 1.4647 |

TABLE 25
Example 12

| Sn | 14 | 15 |
|---|---|---|
| KA | −2.6240447E+04 | 4.2458053E+00 |
| A3 | 1.9254014E−03 | 5.2774084E−03 |
| A4 | 1.4315671E−02 | −1.9039777E−03 |
| A5 | −3.8311026E−02 | 1.2131102E−01 |
| A6 | 7.7670602E−02 | 1.5320564E−02 |
| A7 | 2.4434052E−01 | −3.5916851E−01 |
| A8 | −2.6070028E−01 | −2.7822558E−01 |
| A9 | −2.2610606E+00 | 9.1035885E−01 |
| A10 | 2.3295382E+00 | 1.3130400E+00 |
| A11 | 2.9305627E+00 | −3.7145502E+00 |
| A12 | −3.7514246E+00 | 1.9056204E+00 |

### Example 13

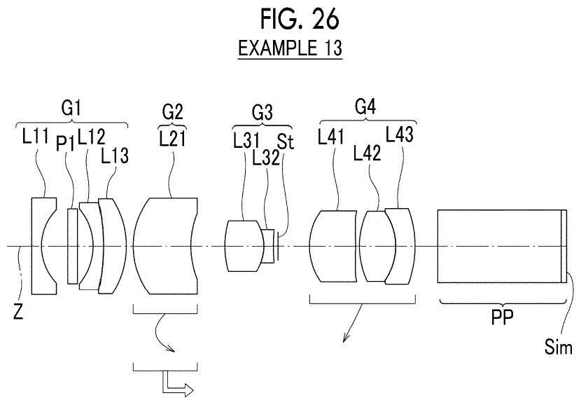

A cross-sectional view of a configuration of an endoscopic objective lens of Example 13 is shown in FIG. 26. The endoscopic objective lens of Example 13 consists of, in order from the object side to the image side, four lens groups including the first lens group G1 having negative optical power, the second lens group G2 having positive optical power, the third lens group G3 having negative optical power, and the fourth lens group G4 having positive optical power.

The first lens group G1 consists of, in order from the object side to the image side, the lens L11, the optical member P1, the lens L12, and the lens L13. The second lens group G2 consists of the lens L21. The third lens group G3 consists of, in order from the object side to the image side, the lens L31, the lens L32, and the aperture stop St. The fourth lens group G4 consists of, in order from the object side to the image side, the lens L41, the lens L42, and the lens L43. The optical member P1 is assumed to be a filter and the like and is a member that has an incident surface and an emission surface parallel to each other, and does not have optical power.

The first lens group G1 corresponds to the first stationary lens group. The second lens group G2 corresponds to the first moving lens group. The third lens group G3 corresponds to the second stationary lens group. The fourth lens group G4 corresponds to the second moving lens group.

During changing the magnification from the wide angle end to the telephoto end, the second lens group G2 moves to the object side and then moves to the image side, the fourth lens group G4 moves to the object side, and other lens groups remain stationary with respect to the image plane Sim. During the focusing from the farthest point observation to the nearest point observation, the second lens group G2 moves to the image side, and other lens groups remain stationary with respect to the image plane Sim.

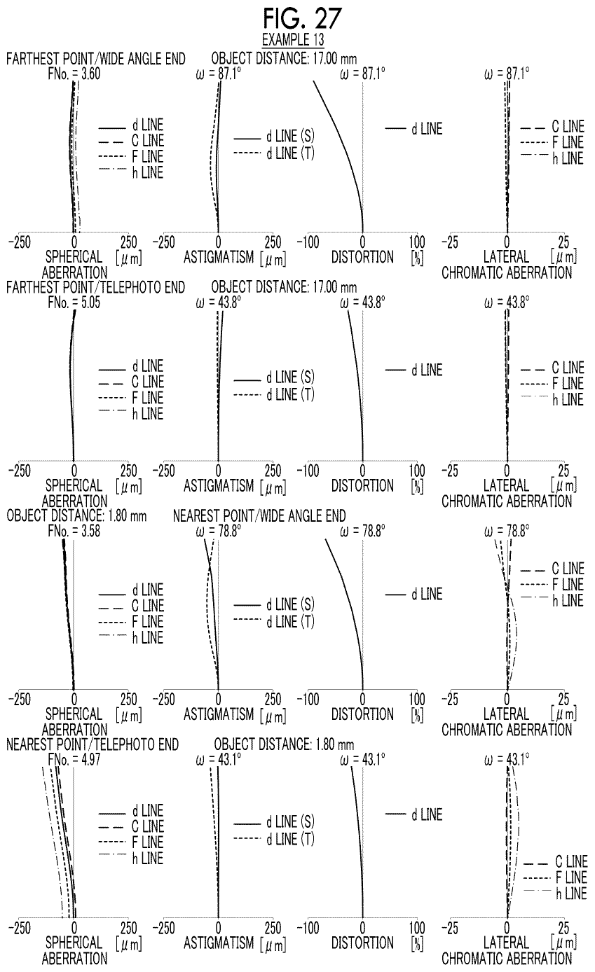

For the endoscopic objective lens of Example 13, Table 26 shows basic lens data, Table 27 shows specifications and variable surface spacings, Table 28 shows aspherical coefficients, and FIG. 27 shows each aberration diagram. Glass described in p. 40 to 42 of the manuscript of the 49th Optical Symposium (duration: June 20 and 21, 2024, host: The Optical Society of Japan, a general incorporated association) can be used as “N231.Glass”, “N181.Glass”, “N200.Glass”, and “N216.Glass” of Material shown in Table 26.

TABLE 26
Example 13

| Sn | R | D | Nd | νd | Material |
|---|---|---|---|---|---|
| 1 | ∞ | 0.2500 | 2.30909 | 17.89 | N231.Glass |
| 2 | 1.3820 | 0.6858 | | | |
| 3 | ∞ | 0.2500 | 1.88300 | 40.76 | S-LAH58.OHARA |
| 4 | ∞ | 0.4067 | | | |
| 5 | −1.5267 | 0.2600 | 1.59522 | 67.73 | S-FPM2.OHARA |
| 6 | −5.5059 | 0.5922 | 1.92286 | 18.90 | S-NPH2.OHARA |
| 7 | −2.3079 | DD[7] | | | |
| 8 | 2.0690 | 1.5000 | 1.81425 | 42.35 | N181.Glass |
| 9 | 2.3518 | DD[9] | | | |
| 10 | 2.3925 | 1.0129 | 1.63980 | 34.47 | S-TIM27.OHARA |
| 11 | −1.2265 | 0.2500 | 2.00266 | 31.67 | N200.Glass |
| 12 | 5.3860 | 0.1175 | | | |
| 13 (St) | ∞ | DD[13] | | | |
| *14 | 1.6567 | 1.1976 | 1.58313 | 59.38 | L-BAL42.OHARA |
| *15 | −10.6074 | 0.1000 | | | |
| 16 | 2.1531 | 0.9436 | 1.43875 | 94.66 | S-FPL55.OHARA |
| 17 | −1.5769 | 0.5045 | 2.16217 | 21.24 | N216.Glass |
| 18 | −2.8279 | DD[18] | | | |
| 19 | ∞ | 3.2000 | 1.92286 | 18.90 | S-NPH2.OHARA |
| 20 | ∞ | 0.1400 | 1.51633 | 64.14 | S-BSL7.OHARA |
| 21 | ∞ | 0.0000 | | | |

TABLE 27
Example 13

| | Farthest Point/ | Farthest Point/ | Nearest Point/ | Nearest Point/ |
|---|---|---|---|---|
| | Wide Angle End | Telephoto End | Wide Angle End | Telephoto End |
| Object Distance | 17.00 | 17.00 | 1.80 | 1.80 |
| Zoom Ratio | 1.00 | 1.39 | 0.99 | 1.30 |
| Focal Length | 0.882 | 1.224 | 0.869 | 1.142 |
| F-Number | 3.60 | 5.05 | 3.58 | 4.97 |
| Maximum Full Angle | 174.2 | 87.6 | 157.6 | 86.2 |
| of View[°] | | | | |
| Maximum Image Height | 0.848 | 0.848 | 0.848 | 0.848 |
| DD[7] | 0.1781 | 0.2555 | 0.9697 | 1.0471 |
| DD[9] | 0.8837 | 0.8063 | 0.0921 | 0.0147 |
| DD[13] | 0.8074 | 0.1177 | 0.8074 | 0.1177 |
| DD[18] | 0.5876 | 1.2773 | 0.5876 | 1.2773 |

TABLE 28
Example 13

| Sn | 14 | 15 |
|---|---|---|
| KA | −3.5915717E+00 | 7.1557213E+01 |
| A4 | 1.4987355E−01 | 5.3322166E−02 |
| A6 | −1.5714202E−01 | 9.7680668E−02 |
| A8 | 3.8615787E−01 | −1.9060587E−01 |
| A10 | −4.3872369E−01 | 3.8714046E−01 |
| A12 | 8.5287688E−02 | −2.3062235E−01 |
| A14 | 2.0149403E−01 | −1.3882819E−01 |
| A16 | −1.0951847E−01 | 1.6393636E−01 |

Table 29 shows the corresponding values of Conditional Expressions (1) to (6) of the endoscopic objective lens of Examples 1 to 13. Table 29 shows values based on a d line. Preferable ranges of the conditional expressions may be set using the corresponding values of the examples shown in Table 29 as upper limit values and lower limit values of the conditional expressions.

TABLE 29

| Expression Number | Conditional Expression | Example 1 | Example 2 | Example 3 | Example 4 | Example 5 |
|---|---|---|---|---|---|---|
| (1) | YFw/(fFw × tan ωFw) | 0.183 | 0.148 | 0.170 | 0.074 | 0.153 |
| (2) | ωFw/ωFt | 1.951 | 1.860 | 1.946 | 1.970 | 1.957 |
| (3) | ωFw/ωCw | 0.952 | 1.071 | 0.976 | 1.089 | 0.996 |
| (4) | (fCw − fFw)/(D0Fw − D0Cw) | −0.003 | −0.001 | −0.004 | −0.001 | −0.001 |
| (5) | \|fL1/fFw\| | 1.577 | 1.631 | 1.627 | 1.657 | 1.400 |
| (6) | \|f1\|/fE | 0.090 | 0.414 | 0.523 | 0.661 | 0.433 |
| Expression Number | Conditional Expression | Example 6 | Example 7 | Example 8 | Example 9 | Example 10 |
| (1) | YFw/(fFw × tan ωFw) | 0.127 | 0.187 | 0.132 | 0.162 | 0.067 |
| (2) | ωFw/ωFt | 1.592 | 1.908 | 1.968 | 1.542 | 1.447 |
| (3) | ωFw/ωCw | 1.061 | 1.016 | 1.079 | 1.076 | 1.094 |
| (4) | (fCw − fFw)/(D0Fw − D0Cw) | −0.003 | −0.003 | −0.002 | 0.002 | −0.002 |
| (5) | \|fL1/fFw\| | 1.080 | 1.407 | 1.421 | 0.874 | 0.939 |
| (6) | \|f1\|/fE | 0.463 | 0.620 | 0.393 | 0.528 | 2.029 |

| Expression Number | Conditional Expression | Example 11 | Example 12 | Example 13 |
|---|---|---|---|---|
| (1) | YFw/(fFw × tan ωFw) | 0.111 | 0.153 | 0.048 |
| (2) | ωFw/ωFt | 2.035 | 2.268 | 1.991 |
| (3) | ωFw/ωCw | 0.938 | 1.016 | 1.106 |
| (4) | (fCw − fFw)/(D0Fw − D0Cw) | −0.004 | −0.002 | −0.001 |
| (5) | \|fL1/fFw\| | 1.421 | 1.864 | 1.198 |
| (6) | \|f1\|/fE | 0.437 | 0.343 | 0.672 |

The endoscopic objective lens of Examples 1 to 13 enables both wide-range observation and magnified observation and can independently perform magnification changing and focusing by causing a moving lens group to behave differently during changing the magnification and during the focusing, while including two or fewer moving lens groups. In addition, the endoscopic objective lens of Examples 1 to 13 maintains high optical performance by favorably correcting various types of aberration in any of four states shown in each aberration diagram.

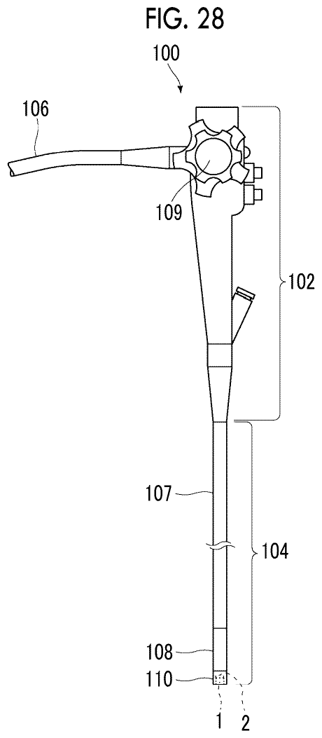

Next, an endoscope according to an embodiment of the present disclosure will be described. FIG. 28 shows a schematic overall configuration diagram of the endoscope according to an embodiment of the present disclosure. An endoscope 100 shown in FIG. 28 mainly comprises an operation part 102, an insertion part 104, and a universal cord 106 connected to a connector part (not shown). Most of the insertion part 104 is a soft part 107 that bends in any direction along an insertion path. A bendable part 108 is connected to a distal end of the soft part 107, and a tip part 110 is connected to a distal end of the bendable part 108. The bendable part 108 is provided to direct the tip part 110 in a desired direction, and a bending operation can be performed by rotationally moving a bending operation knob 109 provided in the operation part 102. An endoscopic objective lens 1 according to an embodiment of the present disclosure and an imaging element 2 are provided at an inner distal end of the tip part 110. For example, a charge coupled device (CCD) or a complementary metal oxide semiconductor (CMOS) can be used as the imaging element 2. The imaging element 2 is disposed such that an imaging surface thereof coincides with an image plane of the endoscopic objective lens 1. FIG. 28 conceptually shows the endoscopic objective lens 1 and the imaging element 2.

While the disclosed technology is described above using embodiments and examples, the disclosed technology is not limited to the embodiments and the examples, and various modifications can be made. For example, a curvature radius, a surface spacing, a refractive index, and an Abbe number of each lens are not limited to the values shown in each numerical value example described above and may have other values.

The following appendices are further disclosed with respect to the embodiments and the examples described above.

### Appendix 1

An endoscopic objective lens consisting of, in order from a position closest to an object side to an image side, four or fewer lens groups including a first stationary lens group having optical power, a first moving lens group having optical power, and a second moving lens group having optical power,
in which during changing magnification from a wide angle end state in farthest point observation to a telephoto end state in the farthest point observation, the first moving lens group and the second moving lens group move along different trajectories from each other,
during focusing from the wide angle end state in the farthest point observation to a wide angle end state in nearest point observation, at least one of the first moving lens group or the second moving lens group moves,
for a lens group that moves during the focusing, a moving amount during changing the magnification from the wide angle end state in the farthest point observation to the telephoto end state in the farthest point observation is different from a moving amount during the focusing from the wide angle end state in the farthest point observation to the wide angle end state in the nearest point observation,
the first stationary lens group remains stationary with respect to an image plane during changing the magnification and during the focusing, and
in a case where a maximum image height in the wide angle end state in the farthest point observation is denoted by YFw,
a maximum half angle of view in the wide angle end state in the farthest point observation is denoted by ωFw, and
a focal length of a whole system in the wide angle end state in the farthest point observation is denoted by fFw,

Conditional Expression (1) is satisfied, which is represented by

0<YFw/(fFw×tan ωFw)<0.5 (1).

### Appendix 2

The endoscopic objective lens according to Appendix 1, in which during the focusing from the wide angle end state in the farthest point observation to the wide angle end state in the nearest point observation, the first moving lens group and the second moving lens group move along different trajectories from each other.

### Appendix 3

The endoscopic objective lens according to Appendix 1, in which during the focusing from the wide angle end state in the farthest point observation to the wide angle end state in the nearest point observation, the first moving lens group and the second moving lens group move in an integrated manner.

### Appendix 4

The endoscopic objective lens according to Appendix 1 or 2, further comprising a second stationary lens group that remains stationary with respect to the image plane during changing the magnification and during the focusing and that has optical power, between the first moving lens group and the second moving lens group.

### Appendix 5

The endoscopic objective lens according to any one of Appendices 1 to 3, further comprising a second stationary lens group that remains stationary with respect to the image plane during changing the magnification and during the focusing and that has optical power, on an image side of the second moving lens group.

### Appendix 6

The endoscopic objective lens according to any one of Appendices 1 to 5, in which in a case where a maximum half angle of view in the telephoto end state in the farthest point observation is denoted by ωFt,

Conditional Expression (2) is satisfied, which is represented by

1.2<ωFw/ωFt<3 (2).

### Appendix 7

The endoscopic objective lens according to any one of Appendices 1 to 6, in which in a case where a maximum half angle of view in the wide angle end state in the nearest point observation is denoted by ωCw,

Conditional Expression (3) is satisfied, which is represented by

0.5<ωFw/ωCw<2 (3).

### Appendix 8

The endoscopic objective lens according to any one of Appendices 1 to 7, in which in a case where a distance on an optical axis from a farthest point in the wide angle end state in the farthest point observation to a surface closest to the object side of the first stationary lens group is denoted by D0Fw,
a focal length of the whole system in the wide angle end state in the nearest point observation is denoted by fCw, and
a distance on the optical axis from a nearest point in the wide angle end state in the nearest point observation to the surface closest to the object side of the first stationary lens group is denoted by D0Cw,

Conditional Expression (4) is satisfied, which is represented by

−0.1<(fCw−fFw)/(D0Fw−D0Cw)<0.1 (4).

### Appendix 9

The endoscopic objective lens according to any one of Appendices 1 to 8, in which the first stationary lens group includes, in order from the object side to the image side, a first negative lens, a second negative lens, and a first positive lens, and the second negative lens and the first positive lens are cemented to each other.

### Appendix 10

The endoscopic objective lens according to Appendix 9, in which a lens surface of the first negative lens on the object side is a plane.

### Appendix 11

The endoscopic objective lens according to Appendix 9 or 10, in which in a case where a focal length of the first negative lens is denoted by fL1, Conditional Expression (5) is satisfied, which is represented by

0.5<|fL1/fFw|<5 (5).

### Appendix 12

The endoscopic objective lens according to any one of Appendices 1 to 11, in which in a case where a focal length of the first stationary lens group is denoted by f1, and
a focal length of a lens group closest to the image side in the endoscopic objective lens is denoted by fE,

Conditional Expression (6) is satisfied, which is represented by

0.05<|f1|/fE<2.3 (6).

### Appendix 13

The endoscopic objective lens according to any one of Appendices 1 to 12, in which Conditional Expression (1-1) is satisfied, which is represented by

0.02<YFw/(fFw×tan ωFw)<0.3 (1-1).

### Appendix 14

The endoscopic objective lens according to any one of Appendices 1 to 12, in which Conditional Expression (1-2) is satisfied, which is represented by

0.04<YFw/(fFw×tan ωFw)<0.2 (1-2).

### Appendix 15

The endoscopic objective lens according to Appendix 7, in which Conditional Expression (3-1) is satisfied, which is represented by

0.9<ωFw/ωCw<1.2 (3-1).

### Appendix 16

The endoscopic objective lens according to Appendix 8, in which Conditional Expression (4-1) is satisfied, which is represented by

−0.01<(fCw−fFw)/(D0Fw−D0Cw)<0.01 (4-1).

### Appendix 17

The endoscopic objective lens according to Appendix 11, in which Conditional Expression (5-1) is satisfied, which is represented by

0.7<|fL1/fFw|<3 (5-1).

### Appendix 18

The endoscopic objective lens according to Appendix 11, in which Conditional Expression (5-2) is satisfied, which is represented by

0.8<|fL1/fFw|<2 (5-2).

### Appendix 19

The endoscopic objective lens according to Appendix 12, in which Conditional Expression (6-1) is satisfied, which is represented by

0.15<|f1|/fE<1.2 (6-1).

### Appendix 20

An endoscope comprising the endoscopic objective lens according to any one of Appendices 1 to 19.

What is claimed is:

1. An endoscopic objective lens consisting of, in order from a position closest to an object side to an image side, four or fewer lens groups including a first stationary lens group having optical power, a first moving lens group having optical power, and a second moving lens group having optical power,
wherein during changing magnification from a wide angle end state in farthest point observation to a telephoto end state in the farthest point observation, the first moving lens group and the second moving lens group move along different trajectories from each other,
during focusing from the wide angle end state in the farthest point observation to a wide angle end state in nearest point observation, at least one of the first moving lens group or the second moving lens group moves,
for a lens group that moves during the focusing, a moving amount during changing the magnification from the wide angle end state in the farthest point observation to the telephoto end state in the farthest point observation is different from a moving amount during the focusing from the wide angle end state in the farthest point observation to the wide angle end state in the nearest point observation,
the first stationary lens group remains stationary with respect to an image plane during changing the magnification and during the focusing, and
in a case where a maximum image height in the wide angle end state in the farthest point observation is denoted by YFw,
a maximum half angle of view in the wide angle end state in the farthest point observation is denoted by ωFw, and
a focal length of the endoscopic objective lens in the wide angle end state in the farthest point observation is denoted by fFw,
Conditional Expression (1) is satisfied, which is represented by
0<YFw/(fFw×tan ωFw)<0.5 (1).

2. The endoscopic objective lens according to claim 1,
wherein during the focusing from the wide angle end state in the farthest point observation to the wide angle end state in the nearest point observation, the first moving lens group and the second moving lens group move along different trajectories from each other.

3. The endoscopic objective lens according to claim 1,
wherein during the focusing from the wide angle end state in the farthest point observation to the wide angle end state in the nearest point observation, the first moving lens group and the second moving lens group move in an integrated manner.

4. The endoscopic objective lens according to claim 1, further comprising:
a second stationary lens group that remains stationary with respect to the image plane during changing the magnification and during the focusing and that has optical power, between the first moving lens group and the second moving lens group.

5. The endoscopic objective lens according to claim 1, further comprising:
a second stationary lens group that remains stationary with respect to the image plane during changing the magnification and during the focusing and that has optical power, on an image side of the second moving lens group.

6. The endoscopic objective lens according to claim 1,
wherein in a case where a maximum half angle of view in the telephoto end state in the farthest point observation is denoted by ωFt,
Conditional Expression (2) is satisfied, which is represented by
1.2<ωFw/ωFt<3 (2).

7. The endoscopic objective lens according to claim 1,
wherein in a case where a maximum half angle of view in the wide angle end state in the nearest point observation is denoted by ωCw,
Conditional Expression (3) is satisfied, which is represented by
0.5<ωFw/ωCw<2 (3).

8. The endoscopic objective lens according to claim 1,
wherein in a case where a distance on an optical axis from a farthest point in the wide angle end state in the farthest point observation to a surface closest to the object side of the first stationary lens group is denoted by D0Fw,
a focal length of the endoscopic objective lens in the wide angle end state in the nearest point observation is denoted by fCw, and
a distance on the optical axis from a nearest point in the wide angle end state in the nearest point observation to the surface closest to the object side of the first stationary lens group is denoted by D0Cw,
Conditional Expression (4) is satisfied, which is represented by
−0.1<(fCw−fFw)/(D0Fw−D0Cw)<0.1 (4).

9. The endoscopic objective lens according to claim 1,
wherein the first stationary lens group includes, in order from the object side to the image side, a first negative lens, a second negative lens, and a first positive lens, and
the second negative lens and the first positive lens are cemented to each other.

10. The endoscopic objective lens according to claim 9,
wherein a lens surface of the first negative lens on the object side is a plane.

11. The endoscopic objective lens according to claim 9,
wherein in a case where a focal length of the first negative lens is denoted by fL1,
Conditional Expression (5) is satisfied, which is represented by
0.5<|fL1/fFw|<5 (5).

12. The endoscopic objective lens according to claim 1,
wherein in a case where a focal length of the first stationary lens group is denoted by f1, and
a focal length of a lens group closest to the image side in the endoscopic objective lens is denoted by fE,
Conditional Expression (6) is satisfied, which is represented by
0.05<|f1|/fE<2.3 (6).

13. The endoscopic objective lens according to claim 1,
wherein Conditional Expression (1-1) is satisfied, which is represented by
0.02<YFw/(fFw×tan ωFw)<0.3 (1-1).

14. The endoscopic objective lens according to claim 1,
wherein Conditional Expression (1-2) is satisfied, which is represented by
0.04<YFw/(fFw×tan ωFw)<0.2 (1-2).

15. The endoscopic objective lens according to claim 7,
wherein Conditional Expression (3-1) is satisfied, which is represented by
0.9<ωFw/ωCw<1.2 (3-1).

16. The endoscopic objective lens according to claim 8,
wherein Conditional Expression (4-1) is satisfied, which is represented by
−0.01<(fCw−fFw)/(D0Fw−D0Cw)<0.01 (4-1).

17. The endoscopic objective lens according to claim 11,
wherein Conditional Expression (5-1) is satisfied, which is represented by
0.7<|fL1/fFw|<3 (5-1).

18. The endoscopic objective lens according to claim 11,
wherein Conditional Expression (5-2) is satisfied, which is represented by
0.8<|fL1/fFw|<2 (5-2).

19. The endoscopic objective lens according to claim 12,
wherein Conditional Expression (6-1) is satisfied, which is represented by
0.15<|f1|/fE<1.2 (6-1).

20. An endoscope comprising:
the endoscopic objective lens according to claim 1.
